(19) United States
(12) Patent Application Publication
(10) Pub. No.: US 2026/0265332 A1
(43) Pub. Date: Sep. 10, 2026

(54) PROCESS FOR PREPARATION OF DUAL GIP/GLP-1 PEPTIDES HAVING CONTROLLED PARTICLE SIZE

(71) Applicant: ASSIA CHEMICAL INDUSTRIES LTD., Tel Aviv (IL)

(72) Inventors: Offir Barel, Kfar Saba (IL); Abed Masarwa, Tayibe (IL); Polina Lapido, Rishon Lezion (IL)

(21) Appl. No.: 19/160,883

(22) PCT Filed: Feb. 29, 2024
(86) PCT No.: PCT/IB2024/051967

§ 371 Date: Aug. 29, 2025

Related U.S. Application Data
(60) Provisional application No. 63/449,378, filed on Mar. 2, 2023.

(51) Int. Cl. C07K 14/605; A61K 9/16; A61K 9/19; A61K 38/00

(52) U.S. Cl. CPC C07K 14/605; A61K 9/1688; A61K 9/19; A61K 38/00

(57) ABSTRACT

The present disclosure relates to processes for preparation of a peptide powder, and the preparation of peptide particles, for example a Dual GIP/GLP-1 peptide such as Tirzepatide. More specifically, the disclosure relates to a process comprising lyophilizing a solution comprising the peptide; and particle size reduction of the obtained lyophilizate. The disclosure further relates to Dual GIP/GLP-1 peptides, particularly Tirzepatide, obtainable by said process and its use in medicine and cosmetic treatment.

### FIELD OF THE DISCLOSURE

The present disclosure relates to processes for preparation of a peptide powder, and the preparation of peptide particles, for example a Dual GIP/GLP-1 peptide such as Tirzepatide. More specifically, the disclosure relates to a process comprising lyophilizing a solution comprising the peptide; and particle size reduction of the obtained lyophilizate. The disclosure further relates to Dual GIP/GLP-1 peptides, particularly Tirzepatide, obtainable by said process and its use in medicine and cosmetic treatment.

### BACKGROUND OF THE DISCLOSURE

Dual GIP/GLP-1 peptides such as Tirzepatide, represent a class of drugs that can be used to treat type 2 diabetes and Obesity.

Synthetic peptide drugs such as Dual GIP/GLP-1 peptides are typically manufactured using multistep solid phase synthesis. The resulting peptides are typically purified by HPLC, and the products isolated primarily by lyophilization or by spray drying in order to remove the chromatography solvents and buffers.

Lyophilization and spray drying are well-known processes used for the solidification of unstable or biopharmaceutical formulations. However, the products (lyophilizates) produced by lyophilization are generally characterized by low bulk density. Lyophilized particles of an active pharmaceutical ingredient (“API”) having low bulk density may display poor or limited flowability, which may ultimately result in difficulties in milling processes, as well as difficulties in blending during the manufacture of a pharmaceutical composition. In the case of peptides such as Dual GIP/GLP-1 peptides, where the drugs are typically administered in relatively low doses, content uniformity in a solid dosage form is of particular importance. Content uniformity is particularly difficult to control in the case of poorly flowable substances and/or substances having unsuitable particle size distribution. However, preparation of a solid dosage form particularly of peptides such as GLP-1 peptides generally require an API having a specific particle size distribution (“PSD”) and other characteristics that cannot be controlled by lyophilization. Thus, prior art lyophilization processes which are typically used in the preparation of peptides such as Dual GIP/GLP-1 peptides result in the production of fluffy and non-flowable materials. The lyophilized products typically do not have the required small particle sizes for the final dosage form in the case of low dose drugs, such as the Dual GIP/GLP-1 peptides, and are not good candidates for particle size reduction steps such as milling or sieving.

The problems associated with lyophilized materials having poor flowability have generally been difficult to resolve. Indeed, the difficulties of processing prior art lyophilizates, which are typically fluffy and non-flowable materials are well known and have investigators have been prompted to seek alternatives to the freeze drying process. This problem is especially acute when the lyophilizates are peptides. A common technology for obtaining powders of, e.g. peptides, is through spray drying processes. However, whilst spray drying is a useful method to form a product which does not suffer from the above-discussed issues of fluffy or non-flowable materials commonly obtained from lyophilization, there are other problems associated with spray drying, particularly in the production of, e.g. peptides.

In particular, spray drying technology requires preparation of a feed solution comprising the API and exposure of the API-containing feed solution to heat, which can be especially detrimental for the API purity over time (for example due to decomposition), especially in large scale during spray drying of drug substances. This problem can be particularly acute in the case of peptides, which are often heat sensitive. Another drawback of the spray drying process is the low yield obtained because of material loss, which is particularly undesirable in the cases of high cost APIs such as peptides. For at least these reasons, there is still a need for additional processes for preparation of APIs such as peptides and particular Dual GIP/GLP-1 peptides having controlled PSDs that will be suitable in large scale production.

It has unexpectedly been found that lyophilizing a solution of a peptide wherein the peptide is present in solution at high concentrations that are greater than typical concentrations advantageously produces peptide powders (i.e. the lyophilizate), wherein the powder is flowable and can readily be processed in subsequent steps such as particle size reduction for example by milling or sieving.

### SUMMARY OF THE DISCLOSURE

The present disclosure relates to a process for the preparation of a peptide powder, preferably wherein the peptide is a Dual GIP/GLP-1, wherein the process comprises: a) providing a solution comprising one or more solvents and a peptide, preferably a Dual GIP/GLP-1 peptide, preferably Tirzepatide, or a salt thereof; and most preferably Tirzepatide; wherein the concentration (w/w) of the peptide in the solution is not less than about 2.5% particularly, the concentration of the peptide may range from: about 2.5% (w/w) to about 40% w/w), about 2.5% (w/w) to about 35% (w/w), about 2.5% to about 30% (w/w), about 2.5% (w/w) to about 25% (w/w), or more particularly about 2.5% (w/w) to about 20% (w/w), or wherein the concentration of the peptide in the solution of step a) is about 3.0 about 20% (w/w), or about 3.5% to about 15% (w/w), or about 3.5% to about 15% (w/w), or about 3.5% to about 15% (w/w), or about 4.0% to about 12% (w/w), or about 4.5% to about 11% (w/w); and wherein the solution has a pH of: about 5 to about 10, preferably to about 6 to about 9, and more preferably about 7 to about 8.5; and b) lyophilizing the solution. The peptides produced by the lyophilization process described herein are advantageously in the form of a powder, particularly a flowable or free-flowing powder. According to any aspect or embodiment of the disclosure, the powder may be subjected to particle size reduction, optionally by milling or by sieving.

In yet another aspect the present disclosure provides a peptide powder, preferably wherein the peptide is a Dual GIP/GLP-1 peptide, preferably Tirzepatide or a salt thereof; and most preferably Tirzepatide, produced by any of the processes disclosed herein.

The present disclosure also provides the use of a peptide powder produced by any of the processes of the disclosure in the preparation of pharmaceutical compositions and/or formulations or dietary compositions, preferably in the preparation of oral dosage forms, more preferably in the preparations of tablets.

The present disclosure also provides a peptide powder produced by the processes of the disclosure for use in the preparation of pharmaceutical compositions and/or formulations, or dietary compositions, preferably in the preparation of oral dosage forms, more preferably in the preparations of tablets.

The present disclosure further includes processes for the preparation of the above mentioned pharmaceutical compositions, pharmaceutical formulations, or dietary compositions, comprising a peptide, preferably a Dual GIP/GLP-1 peptide, preferably Tirzepatide or a salt thereof, and most preferably Tirzepatide; wherein the process comprises combining the peptide powder obtained by any one of the processes of the present disclosure with at least one pharmaceutically acceptable, or dietary acceptable excipient.

The peptide powder produced according to the processes described herein and the pharmaceutical compositions or formulations thereof may be used as medicaments, such as for the treatment of type 2 diabetes, overweight or obesity, and preferably type 2 diabetes or for the treatment of: Renal Insufficiency, End Stage Renal Disease, Hepatic Insufficiency, Wolfram Syndrome, Type II Diabetes, Atherosclerosis, Knee Osteoarthritis, Heart Failure With Preserved Ejection Fraction, Obesity and Chronic Kidney Disease, Obstructive Sleep Apnea, Non-Alcoholic Fatty Liver Disease, or Non-alcoholic Steatohepatitis with Type 2 Diabetes.

The peptide powder produced according to the processes described herein and the dietary compositions thereof may be used for cosmetic weight loss in non-overweight non-obese subjects.

The present disclosure also provides methods for the treatment of patients suffering from type 2 diabetes, overweight or obesity, and preferably type 2 diabetes, or patients suffering from: Renal Insufficiency, End Stage Renal Disease, Hepatic Insufficiency, Wolfram Syndrome, Type II Diabetes, Atherosclerosis, Knee Osteoarthritis, Heart Failure With Preserved Ejection Fraction, Obesity and Chronic Kidney Disease, Obstructive Sleep Apnea, Non-Alcoholic Fatty Liver Disease, or Non-alcoholic Steatohepatitis with Type 2 Diabetes, by administering a therapeutically effective amount of a peptide powder produced according to the processes described herein, or a pharmaceutical composition thereof, to a subject suffering from type 2 diabetes, overweight or obesity, and preferably type 2 diabetes, or a subject suffering from: Renal Insufficiency, End Stage Renal Disease, Hepatic Insufficiency, Wolfram Syndrome, Type II Diabetes, Atherosclerosis, Knee Osteoarthritis, Heart Failure With Preserved Ejection Fraction, Obesity and Chronic Kidney Disease, Obstructive Sleep Apnea, Non-Alcoholic Fatty Liver Disease, or Non-alcoholic Steatohepatitis with Type 2 Diabetes, or otherwise in need of the treatment.

The present disclosure further provides methods for cosmetic weight loss in non-overweight non-obese subjects by administering an effective amount of a peptide powder produced according to the processes described herein, or a dietary composition thereof.

The present disclosure also provides uses of a peptide powder obtainable by the methods of the present disclosure or at least one of the above pharmaceutical compositions thereof, for the manufacture of medicaments for treating type 2 diabetes, overweight or obesity and preferably type 2 diabetes or for the manufacture of a medicament for treating: Renal Insufficiency, End Stage Renal Disease, Hepatic Insufficiency, Wolfram Syndrome, Type II Diabetes, Atherosclerosis, Knee Osteoarthritis, Heart Failure With Preserved Ejection Fraction, Obesity and Chronic Kidney Disease, Obstructive Sleep Apnea, Non-Alcoholic Fatty Liver Disease, or Non-alcoholic Steatohepatitis with Type 2 Diabetes.

The present disclosure also provides uses of a peptide powder obtainable by the methods of the present disclosure or at least one dietary composition thereof, for cosmetic weight loss in non-overweight non-obese subjects.

The present disclosure further relates to processes for the preparation of a peptide particles, preferably wherein the peptide is a Dual GIP/GLP-1 peptide, preferably Tirzepatide or a salt thereof, and most preferably Tirzepatide; wherein the process comprises: a) providing a solution comprising one or more solvents and a peptide, wherein the concentration of the peptide in the solution is not less than about 2.5% w/w; and wherein the solution has a pH of: about 5 to about 10, preferably about 6 to about 9, and more preferably about 7 to about 8.5; and b) lyophilizing the solution; and c) particle size reduction of the product of step b). The maximum concentration (w/w) of the peptide may be just below the saturation concentration of the peptide in the solvent. The concentration of the peptide may particularly range from: about 2.5% (w/w) to about 40% w/w), about 2.5% (w/w) to about 350% (w/w), about 2.5% to about 30% (w/w), about 2.5% (w/w) to about 25% (w/w), or about 2.5% (w/w) to about 20% (w/w) or the concentration of the peptide is about 3.0 about 20% (w/w), or about 3.5% to about 15% (w/w), or about 3.5% to about 15% (w/w), or about 3.5% to about 15% (w/w), or about 4.0% to about 12% (w/w), or about 4.5% to about 11% (w/w). Preferably, the concentration of the peptide in the solution is: about 2.5% (w/w) to about 20% (w/w). Preferably, the particle size reduction in step (c) is carried out to produce particles having a particle size suitable for including in a pharmaceutical formulation, preferably in an oral formulation.

Thus to any aspect or embodiment of the disclosed process, step (c) may be carried out to produce particles having a PSD characterized by a D(90) value of not more than 120 μm±20%, not more than 100 μm±20%, not more than 80 μm±20%, not more than 60 μm±20%, or to produce particle having a PSD characterized by a D(90) value of: not more than 40 μm±20%, preferably not more than 30 μm±20%, or a PSD characterized by a D(90) value of D(90) of: about 8 μm to about 80 μm, about 10 μm to about 70 μm, about 15 μm to about 65 μm, about 20 μm to about 60 μm, or about 20 μm to about 40 μm, wherein the concentration (w/w) of the peptide in step a) is not less than about 2.5% and as high as saturation of the peptide in the solvent or mixture of solvents. Particularly the concentration (w/w) of the peptide is: about 2.5% (w/w) to about 40% w/w), about 2.5% (w/w) to about 350% (w/w), about 2.5% to about 30% (w/w), about 2.5% (w/w) to about 25% (w/w), or about 2.5% (w/w) to about 20% (w/w). More preferably, the concentration of the peptide is from about 2.5% (w/w) to about 20% (w/w) or wherein the concentration of the peptide in the solution of step a) is about 3.0 about 20% (w/w), or about 3.5% to about 15% (w/w), or about 3.5% to about 15% (w/w), or about 3.5% to about 15% (w/w), or about 4.0% to about 12% (w/w), or about 4.5% to about 11% (w/w).

According to any aspect or embodiment of the disclosed process, the particles obtained in step (c) may have a PSD of: not more than 120 μm±20%, not more than 100 μm±20%, not more than 80 μm±20%, not more than 60 μm±20%, preferably the particles obtained in step (c) may have a PSD characterized by a D(90) of: about 8 μm to about 80 μm, about 10 μm to about 70 μm, about 15 μm to about 65 μm, about 20 μm to about 60 μm, or about 20 to about 40 μm.

In yet another aspect the disclosure further relates to processes for preparation of a peptide particles, preferably wherein the peptide is a Dual GIP/GLP-1 peptide, preferably Tirzepatide or a salt thereof; and most preferably Tirzepatide; wherein the process comprises: providing a lyophilized peptide powder, preferably a Dual GIP/GLP-1 peptide, preferably Tirzepatide or a salt thereof, and most preferably Tirzepatide, wherein the peptide is obtained by steps (a)-(b) according the process of the present disclosure; and reducing the particle size to obtain a powder having a PSD characterized by a D(90) value of not more than 120 μm 20%, not more than 100 μm±20%, not more than 80 μm±20%, not more than 60 μm±20%, or to obtain a powder having a PSD characterized by a D(90) value of: not more than 40 μm±20%, preferably not more than 30 μm±20%; or a D(90) of: about 8 μm to about 80 μm, about 10 μm to about 70 μm, about 15 μm to about 65 μm, about 20 μm to about 60 μm, or about 20 to about 40 μm. Preferably the solution in step (a) has concentration of the peptide of from about 2.5% (w/w) to about 20% (w/w).

In yet another aspect the disclosure further relates to processes for preparation of Tirzepatide or salt thereof, preferably Tirzepatide; wherein the particles have a PSD characterized by a D(90) value of: not more than 120 μm±20%, not more than 100 μm 20%, not more than 80 μm±20%, not more than 60 μm±20%, or a PSD characterized by a D(90) value of: not more than 40 μm±20%, preferably not more than 30 μm±20%; or a D(90) of: about 8 μm to about 80 μm, about 10 μm to about 70 μm, about 15 μm to about 65 μm, about 20 μm to about 60 μm, or about 20 to about 40 μm, wherein the process comprises: preparing a peptide powder by a process according to any of steps (a)-(b) as described herein; and subjecting the lyophilized peptide powder to particle size reduction.

In yet another aspect the present disclosure provides peptide particles, preferably Dual GIP/GLP-1 peptide particles, preferably Tirzepatide or a salt thereof, and most preferably Tirzepatide; having a PSD characterized by a D(90) value of not more than 120 μm±20%, not more than 100 μm±20%, not more than 80 μm±20%, not more than 60 μm±20%, or a PSD characterized by a D(90) value of: not more than 40 μm±20%, preferably not more than 30 μm±20%; or a PSD characterized by a D(90) value of: not more than 40 μm±20%, preferably not more than 30 μm±20%; or a D(90) value of: about 8 μm to about 80 μm, about 10 μm to about 70 μm, about 15 μm to about 65 μm, about 20 μm to about 60 μm, or about 20 to about 40 μm, obtainable by a process according to any aspect or embodiment of the disclosure.

The present disclosure also provides the use of peptide particles, preferably Dual GIP/GLP-1 peptide particles, preferably Tirzepatide or a salt thereof; and most preferably Tirzepatide; having a PSD characterized by a D(90) value of not more than 120 μm±20%, not more than 100 μm±20%, not more than 80 μm±20%, not more than 60 μm±20%, or a PSD characterized by a D(90) value of: not more than 40 μm±20%, preferably not more than 30 μm±20%; or a D(90) value of: about 8 μm to about 80 μm, about 10 μm to about 70 μm, about 15 μm to about 65 μm, about 20 μm to about 60 μm, or about 20 to about 40 μm, obtainable by the processes of the disclosure in the preparation of pharmaceutical compositions and/or formulations or dietary compositions.

The present disclosure also provides peptide particles, preferably Dual GIP/GLP-1 peptide particles, preferably Tirzepatide or a salt thereof; and most preferably Tirzepatide; having a PSD characterized by a D(90) value of not more than 120 μm±20%, not more than 100 μm±20%, not more than 80 μm±20%, not more than 60 μm±20%, or a PSD characterized by a D(90) value of: not more than 40 μm±20%, preferably not more than 30 μm±20%; or a D(90) value of: about 8 μm to about 80 μm, about 10 μm to about 70 μm, about 15 μm to about 65 μm, about 20 μm to about 60 μm, or about 20 to about 40 μm, obtainable by any of the processes of the disclosure for use in the preparation of pharmaceutical compositions and/or formulations or dietary compositions.

The present disclosure further includes processes for the preparation of the above mentioned pharmaceutical composition or pharmaceutical formulation, or dietary composition, preferably for oral administration and more preferably wherein the pharmaceutical formulation is a tablet a Dual GIP/GLP-1 peptide, preferably Tirzepatide or a salt thereof, and most preferably Tirzepatide; wherein the process comprises combining the Dual GIP/GLP-1 peptide particles, preferably Tirzepatide or a salt thereof, and most preferably Tirzepatide; having a PSD characterized by a D(90) value of not more than 120 μm±20%, not more than 100 μm±20%, not more than 80 μm±20%, not more than 60 μm±20%, or a PSD characterized by a D(90) value of: not more than 40 μm±20%, preferably not more than 30 μm±20% particles; or a D(90) value of: about 8 μm to about 80 μm, about 10 μm to about 70 μm, about 15 μm to about 65 μm, about 20 μm to about 60 μm, or about 20 to about 40 μm, obtainable by any of the processes of the present disclosure with at least one pharmaceutically acceptable excipient.

The present disclosure provides Dual GIP/GLP-1 peptide particles, preferably Tirzepatide or a salt thereof, and most preferably Tirzepatide; having a PSD characterized by a D(90) value of not more than 120 μm±20%, not more than 100 μm±20%, not more than 80 μm±20%, not more than 60 μm±20%, or a PSD characterized by a D(90) value of: not more than 40 μm±20%, preferably not more than 30 μm±20%; or a D(90) value of: about 8 μm to about 80 μm, about 10 μm to about 70 μm, about 15 μm to about 65 μm, about 20 μm to about 60 μm, or about 20 to about 40 μm, obtainable according to any of the processes described herein and the pharmaceutical compositions or formulations, may be used as medicaments, such as for the treatment of type 2 diabetes, overweight or obesity, and preferably type 2 diabetes, or for the treatment of: Renal Insufficiency, End Stage Renal Disease, Hepatic Insufficiency, Wolfram Syndrome, Type II Diabetes, Atherosclerosis, Knee Osteoarthritis, Heart Failure With Preserved Ejection Fraction, Obesity and Chronic Kidney Disease, Obstructive Sleep Apnea, Non-Alcoholic Fatty Liver Disease, or Non-alcoholic Steatohepatitis with Type 2 Diabetes.

The present disclosure provides Dual GIP/GLP-1 peptide particles, preferably Tirzepatide or a salt thereof, and most preferably Tirzepatide; having a PSD characterized by a D(90) value of not more than 120 μm±20%, not more than 100 μm±20%, not more than 80 μm±20%, not more than 60 μm±20%, or a PSD characterized by a D(90) value of: not more than 40 μm±20%, preferably not more than 30 μm±20%; or a D(90) value of: about 8 μm to about 80 μm, about 10 μm to about 70 μm, about 15 μm to about 65 μm, about 20 μm to about 60 μm, or about 20 to about 40 μm, obtainable according to any of the processes described herein and the compositions or formulations comprising the Dual GIP/GLP-1 peptide particles having a PSD characterized by a D(90) value of not more than 120 μm 20%, not more than 100 μm±20%, not more than 80 μm±20%, not more than 60 μm±20%, or a PSD characterized by a D(90) value of: not more than 40 μm±20%, preferably not more than 30 μm±20%; or a D(90) value of: about 8 μm to about 80 μm, about 10 μm to about 70 μm, about 15 μm to about 65 μm, about 20 μm to about 60 μm, or about 20 to about 40 μm, obtainable according to any of the processes described herein for cosmetic weight loss in non-overweight non-obese subjects.

The present disclosure also provides methods for the treatment of type 2 diabetes, overweight or obesity, and preferably type 2 diabetes, or for the treatment of: Renal Insufficiency, End Stage Renal Disease, Hepatic Insufficiency, Wolfram Syndrome, Type II Diabetes, Atherosclerosis, Knee Osteoarthritis, Heart Failure With Preserved Ejection Fraction, Obesity and Chronic Kidney Disease, Obstructive Sleep Apnea, Non-Alcoholic Fatty Liver Disease, or Non-alcoholic Steatohepatitis with Type 2 Diabetes, by administering a therapeutically effective amount of Dual GIP/GLP-1 peptide particles, preferably Tirzepatide or a salt thereof, and most preferably Tirzepatide; having a PSD characterized by a D(90) value of not more than 120 μm±20%, not more than 100 μm±20%, not more than 80 μm±20%, not more than 60 μm±20%, or a PSD characterized by a D(90) value of: not more than 40 μm±20%, preferably not more than 30 μm±20% or a D(90) value of: about 8 μm to about 80 μm, about 10 μm to about 70 μm, about 15 μm to about 65 μm, about 20 μm to about 60 μm, or about 20 to about 40 μm, obtainable according to any of the processes described herein, or at least one of the above pharmaceutical compositions or pharmaceutical formulation, to a subject suffering from type 2 diabetes, overweight or obesity, and preferably type 2 diabetes, or to a subject suffering from: Renal Insufficiency, End Stage Renal Disease, Hepatic Insufficiency, Wolfram Syndrome, Type II Diabetes, Atherosclerosis, Knee Osteoarthritis, Heart Failure With Preserved Ejection Fraction, Obesity and Chronic Kidney Disease, Obstructive Sleep Apnea, Non-Alcoholic Fatty Liver Disease, or Non-alcoholic Steatohepatitis with Type 2 Diabetes, or otherwise in need of the treatment.

The present disclosure further provides methods for cosmetic weight loss in non-overweight non-obese subjects comprising administering an effective amount of Dual GIP/GLP-1 peptide particles, preferably Tirzepatide or a salt thereof, and most preferably Tirzepatide; having a PSD characterized by a D(90) value of not more than 120 μm±20%, not more than 100 μm±20%, not more than 80 μm±20%, not more than 60 μm±20%, or a PSD characterized by a D(90) value of: not more than 40 μm±20%, preferably not more than 30 μm±20% or a D(90) value of: about 8 μm to about 80 μm, about 10 μm to about 70 μm, about 15 μm to about 65 μm, about 20 μm to about 60 μm, or about 20 to about 40 μm, obtainable according to any of the processes described herein.

The present disclosure also provides uses of Dual GIP/GLP-1 peptide particles, preferably Tirzepatide or a salt thereof; and most preferably Tirzepatide; having a PSD characterized by a D(90) value of not more than 120 μm±20%, not more than 100 μm 20%, not more than 80 μm±20%, not more than 60 μm±20%, or a PSD characterized by a D(90) value of: not more than 40 μm±20%, preferably not more than 30 μm±20% or a D(90) value of: about 8 μm to about 80 μm, about 10 μm to about 70 μm, about 15 μm to about 65 μm, about 20 μm to about 60 μm, or about 20 to about 40 μm, obtainable by the processes of the present disclosure or at least one of the above pharmaceutical compositions, for the manufacture of medicaments for treating type 2 diabetes, overweight or obesity and preferably type 2 diabetes, or for treating: Renal Insufficiency, End Stage Renal Disease, Hepatic Insufficiency, Wolfram Syndrome, Type II Diabetes, Atherosclerosis, Knee Osteoarthritis, Heart Failure With Preserved Ejection Fraction, Obesity and Chronic Kidney Disease, Obstructive Sleep Apnea, Non-Alcoholic Fatty Liver Disease, or Non-alcoholic Steatohepatitis with Type 2 Diabetes.

The present disclosure also provides uses of Dual GIP/GLP-1 peptide particles, preferably Tirzepatide or a salt thereof; and most preferably Tirzepatide; having a PSD characterized by a D(90) value of not more than 120 μm±20%, not more than 100 μm 20%, not more than 80 μm±20%, not more than 60 μm±20%, or a PSD characterized by a D(90) value of: not more than 40 μm±20%, preferably not more than 30 μm±20% or a D(90) value of: about 8 μm to about 80 μm, about 10 μm to about 70 μm, about 15 μm to about 65 μm, about 20 μm to about 60 μm, or about 20 to about 40 μm, obtainable by any of the processes of present disclosure or at least one of the above pharmaceutical compositions, for the manufacture of compositions of formulations for cosmetic weight loss, i.e. weight loss in non-overweight, non-obese subjects.

### BRIEF DESCRIPTION OF THE DRAWINGS

FIG. 1 shows scanning electron microscopy images of the micronized particles prepared according to Example 2: ×750 (A); ×1500 (B)

FIG. 2 shows scanning electron microscopy images of the sieved particles prepared according to Example 3: ×750 (A); ×1500 (B)

FIG. 3 shows scanning electron microscopy images of the sieved particles prepared according to Example 3A: ×300

FIG. 4 shows scanning electron microscopy images of the sieved particles prepared according to Example 3B: ×300

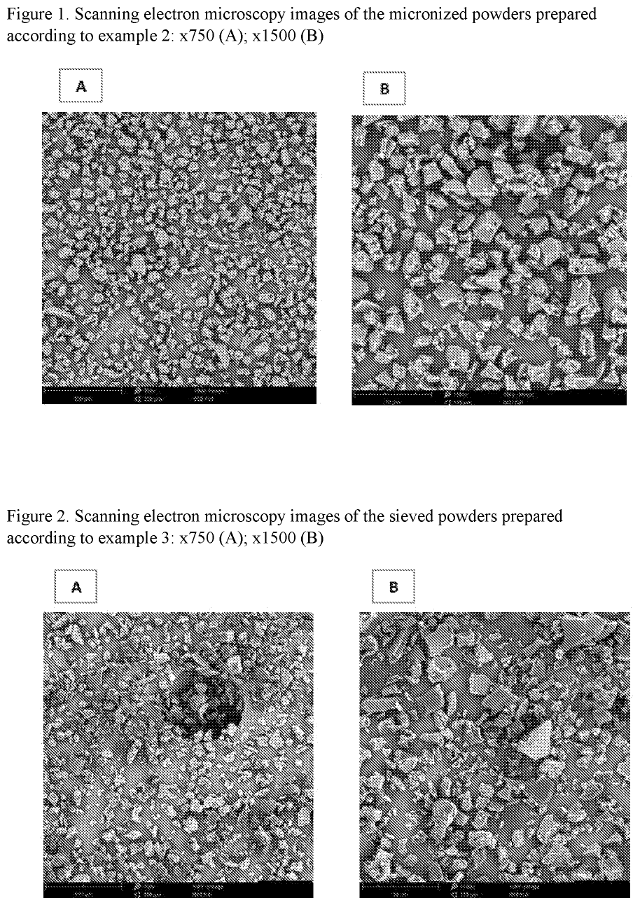

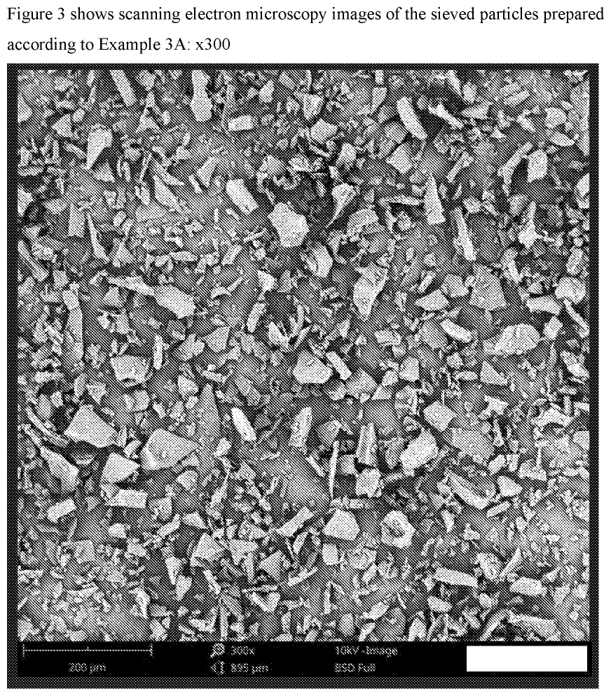

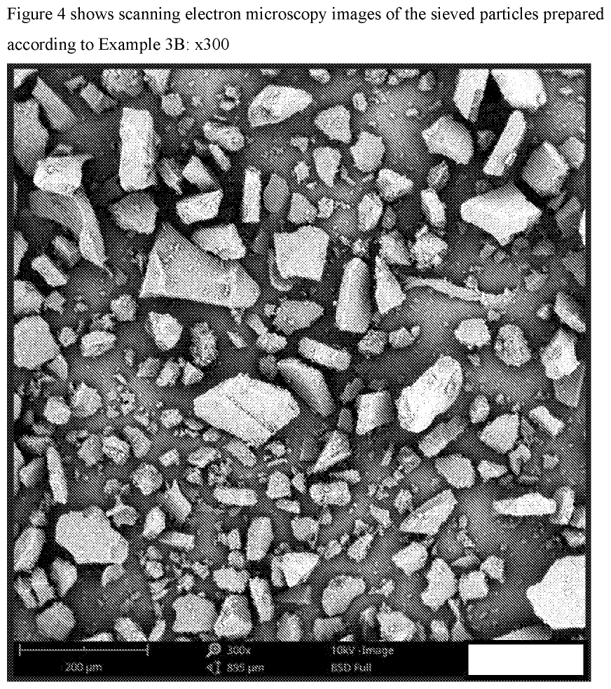

### DETAILED DESCRIPTION OF THE DISCLOSURE

As used herein, the term “room temperature” refers to a temperature of about 20° C. to about 30° C., about 22° C. to about 27° C., or about 25° C.

As used herein, the term “dual GLP-1/GIP receptor agonists” refers to dual incretin peptide mimetic compounds that agonize receptors for both glucagon-like peptide-1 (GLP-1) and glucose-dependent insulinotropic peptide (GIP) receptor agonists such as Tirzepatide.

As used herein, particle size distribution is determined by means of laser diffractometry. More specifically, unless otherwise indicated, the particle size was determined using a Mastersizer 3000 from Malvern Instruments. Preferably, particle size and particle size distribution are determined by laser light diffraction, using Mie theory, and Isopar-G dispersant (isoparaffinic fluid (CAS No) 64742-48-9)) refractive index 1.42.

It will be understood that the term “D(90)” as used herein refers to a particle size distribution where 90% of the volume of particles have a diameter lower than the D(90) value.

As used herein, reference to a (w/w) concentration is to be understood as a reference to concentration based on the weight of the solution of peptide and solvent(s).

As used herein, obesity encompasses obese and severely obese classes of subjects. Obesity is defined in adult humans as a BMI of 30 to 39.9. Severely obese is defined in adult humans as a BMI of 40 or above.

As used herein, overweight is defined in adult humans as a BMI of 25 to 29.9.

As used herein, cosmetic weight loss is understood to mean weight loss in non-overweight, non-obese subjects. For example, cosmetic weight loss optionally refers weight loss in subjects having a body mass index (BMI) of: 18.5 to 24.9, 19 to 24, 20 to 23.5, or 21.0 to 23.

It was surprisingly found that solutions that are lyophilized in a process according to the present disclosure exhibit improved flowability. It was found that increasing the peptide concentration in the solution to be lyophilized positively affects the morphology of the dry material obtained after lyophilization and thicker irregular material is obtained. Further, the flowable powder obtained by the lyophilization process of the present disclosure is easier to handle, suitable for large scale production processes, and may more readily undergo further particle size reduction processes, such as micronizing by a jet mill or sieving to obtain particles having controlled PSD while maintaining the purity of the API. Reduction of particle size by agitative sieving is particularly advantageous for large scale processes. The process of Particle size reduction by Sieving has a very short cycle time in production, it does not involve additional solvents, has low energy consumption and high yield. Therefore the processes of the present disclosure allow preparation of material having any desired particle size distribution by preparation of the flowable powder and further processing the powder to the required PSD.

The present disclosure relates to processes for preparation of peptide powder, preferably dual GLP-1/GIP peptide particles, preferably Tirzepatide or a salt thereof; and most preferably Tirzepatide; wherein the process comprises: a) providing a solution comprising one or more solvents and a peptide, preferably a dual GLP-1/GIP peptide, preferably Tirzepatide or a salt thereof; and most preferably Tirzepatide; wherein the concentration (w/w) of the peptide is not less than about 2.5%, about 2.5% (w/w) to about 40% w/w), about 2.5% (w/w) to about 35% (w/w), about 2.5% to about 30% (w/w), about 2.5% (w/w) to about 25% (w/w), preferably wherein the concentration of the peptide is from about 2.5% (w/w) to about 20% (w/w) or more particularly about 2.5% (w/w) to about 20% (w/w), or wherein the concentration of the peptide in the solution of step a) is about 3.0 about 20% (w/w), or about 3.5% to about 15% (w/w), or about 3.5% to about 15% (w/w), or about 3.5% to about 15% (w/w), or about 4.0% to about 12% (w/w), or about 4.5% to about 11% (w/w); and wherein the solution has a pH of: about 5 to about 10, preferably about 6 to about 9, and more preferably about 7 to about 8.5; and b) lyophilizing the solution.

In any aspect or embodiment, the solvents in step a) may be any solvent or a combination of solvents having a freezing point above −70° C. and that are suitable for lyophilization, i.e. a solvent that sublimes below the triple point at a given minimal pressure, for example 10 pbar. In embodiments the solvents in step a) may be one or more solvents selected from the list consisting of acetic acid, acetone, dimethyl sulfoxide (DMSO), ethanol, water, isopropanol (IPA), methanol, menthol, TFA (Trifluoroacetic acid), and acetonitrile. Preferably, the solvent in step a) may be one or more selected from the group consisting of acetonitrile, IPA, ethanol and water, more preferably the solvent in step a) comprises water. In particular embodiments the solvent in step a) may comprise water and up to about 60% (w/w), preferably up to about 40% (w/w) of any one or a mixture of acetonitrile, IPA and ethanol. Particularly, according to any aspect or embodiment of the disclosure, the solvent comprises water and about 0.5% (w/w) to about 50% (w/w) of acetonitrile, 5% (w/w) to about 30% (w/w) of acetonitrile, more preferably the solvent in step a) comprises water and about 20% (w/w) of acetonitrile. In particularly preferred embodiments, the one or more solvents in step a) consists essentially of (and preferably consists of) acetonitrile and water, preferably wherein acetonitrile is present at: about 0.5% (w/w) to about 50% (w/w), about 1.5% (w/w) to about 45% (w/w), about 2.0% (w/w) to about 40% (w/w), about 2.5% (w/w) to about 30% (w/w); or the acetonitrile is present at: about 5% (w/w) to about 30% (w/w), or about 5% (w/w) to about 25% (w/w), more preferably about 5% (w/w) to about 20% (w/w), particularly about 15% (w/w) to about 20% (w/w), and most preferably about 19% (w/w); or at about 0.5% (w/w) to about 50% (w/w), about 1.5% (w/w) to about 45% (w/w), about 2.0% (w/w) to about 40% (w/w), about 2.5% (w/w) to about 30% (w/w); or about 1.0% (w/w) to about 20% (w/w), about 1.5% (w/w) to about 20% (w/w), about 2.0% (w/w) to about 20% (w/w), about 2.5% (w/w) to about 20% (w/w).

In any aspect or embodiment in step a) the solution may have a pH of: about 6 to about 9, or about 6 to about 8.5, more preferably about 7 to about 8.5, or more preferably to about 7.3 or about 8.

In any aspect or embodiment of the processes disclosed herein, if the solution of peptide in the one or more solvents does not have a pH within the specified range, the pH of the solution may be adjusted by the use of an acidic or basic, typically a basic, agent. Thus, according to any aspect or embodiment of the processes disclosed herein, if the solution of peptide in the one or more solvents does not have a pH within the specified range, the pH of the solution may be adjusted by the use of an acidic or basic, typically a basic, agent. Thus, according to any aspect or embodiment, the pH may be adjusted to the specified range by any suitable basic agent such as but not limited to sodium bicarbonate, ammonium acetate, phosphate buffer, ammonium hydroxide or NaOH. In embodiments the pH is adjusted by ammonium hydroxide, preferably 25% (w/w) solution of ammonium hydroxide or NaOH, to provide the solution in step (a) having a pH within the specified range.

In any aspect or embodiment, step (a) may be performed at any suitable temperature, typically at a temperature of about 15° C. to about 40° C., or about 15° C. to about 30° C., and preferably at room temperature; or wherein step (a) is performed at a temperature of: about 15° C. to about 35° C., about 18° C. to about 25° C., or about 18° C. to about 22° C., or about 20° C.

In any aspect or embodiment of the processes disclosed herein, the solution in step (a) may filtered prior to step (b), preferably wherein the filtration is carried out using a 0.2 micron filter.

In any aspect or embodiment of the processes disclosed herein, the freeze drying step (b) may be carried at a temperature of: about −45° C. to about 30° C., about −40° C. to about 25° C., about −40° C. to about 25° C., or about −35° C. to about 20° C. Thus, the solution in step (a), optionally after a filtration step as discussed above, is cooled to a suitable freeze-drying temperature (preferably: about −15° C. to about −45° C., about −20° C. to about −45° C., about −25° C. to about −40° C., about −30° C. to about −40° C., or about −35° C. The mixture may then be warmed, preferably to a temperature of about 20° C. to about 30° C., about 20° C. to about 25° C., or about 20° C. to about 22° C., or about 20° C., whereupon the solvent(s) is removed by sublimation.

In any aspect or embodiment of the processes disclosed herein, after the freeze drying step, the product may be subjected to a further drying step. The further drying step may be carried out at a temperature of: about 15° C. to about 35° C., about 18° C. to about 25° C., or about 18° C. to about 22° C., or about 20° C. The further drying step may be carried out at reduced pressure, preferably at a pressure of: about 100 pbar to about 800 pbar, about 150 pbar to about 700 pbar, about 180 to about 650 pbar, or about 200 to about 600 pbar.

In embodiments the disclosure relates to processes for preparation of peptide powder, preferably dual GLP-1/GIP peptide particles, preferably Tirzepatide or a salt thereof; and most preferably Tirzepatide; wherein the process comprises a) providing a solution comprising one or more solvents and a peptide, preferably a dual GLP-1/GIP peptide, preferably Tirzepatide or a salt thereof; and most preferably Tirzepatide; wherein the concentration of the peptide is from about 2.5% (w/w) to about 20% (w/w), and wherein the solvents are selected from the list consisting of acetic acid, acetone, dimethyl sulfoxide (DMSO), ethanol, water, isopropanol (IPA), methanol, menthol, TFA, and acetonitrile, preferably, from the group consisting of acetonitrile, IPA, ethanol and water; and wherein the solution has a pH of: about 5 to about 10, about 6 to about 9, about 7 to about 8.5; and b) lyophilizing the solution.

In embodiments the disclosure relates to processes for preparation of peptide powder, preferably dual GLP-1/GIP peptide particles, preferably Tirzepatide or a salt thereof; and most preferably Tirzepatide; wherein the process comprises a) providing a solution comprising one or more solvents and a peptide, preferably a dual GLP-1/GIP peptide, preferably Tirzepatide or a salt thereof; and most preferably Tirzepatide; wherein the concentration of the peptide is from about 2.5% (w/w) to about 20% (w/w), and wherein the solvent in step a) comprises water and up to about 60% (w/w), preferably up to about 40% (w/w), of any one or a mixture of acetonitrile, IPA and ethanol; wherein the solution has a pH of: about 5 to about 10, about 6 to about 9, about 7 to about 8.5; and b) lyophilizing the solution.

In embodiments the disclosure relates to processes for preparation of peptide powder, preferably dual GLP-1/GIP peptide powder, preferably Tirzepatide or a salt thereof; and most preferably Tirzepatide; wherein the process comprises a) providing a solution comprising one or more solvents and a peptide, preferably a dual GLP-1/GIP peptide, preferably Tirzepatide or a salt thereof; and most preferably Tirzepatide; wherein the concentration of the peptide is from about 3% (w/w) to about 15% (w/w), and wherein the solvent in step a) comprises water and up to about 60% (w/w), preferably up to about 40% (w/w), of any one or a mixture of acetonitrile, IPA and ethanol; wherein the solution has a pH of: about 5 to about 10, about 6 to about 9, about 7 to about 8.5; and b) lyophilizing the solution.

In embodiments the disclosure relates to processes for preparation of a peptide powder, preferably dual GLP-1/GIP peptide powder, preferably Tirzepatide or a salt thereof; and most preferably Tirzepatide; wherein the process comprises a) providing a solution comprising one or more solvents and a peptide, preferably a dual GLP-1/GIP peptide, preferably Tirzepatide or a salt thereof; and most preferably Tirzepatide; wherein the concentration of the peptide is from about 2.5% (w/w) to about 20% (w/w) and wherein the solvent in step a) comprises water and about 15% (w/w) to about 30% (w/w) of acetonitrile, more preferably water and about 20% (w/w) of acetonitrile; and wherein the solution has a pH of about 5 to about 10, about 6 to about 9, about 7 to about 8.5; and b) lyophilizing the solution.

In embodiments the disclosure relates to processes for preparation of peptide powder, preferably dual GLP-1/GIP peptide powder, preferably Tirzepatide or a salt thereof; and most preferably Tirzepatide; wherein the process comprises a) providing a solution comprising one or more solvents and a peptide, preferably a dual GLP-1/GIP peptide, preferably Tirzepatide or a salt thereof; and most preferably Tirzepatide; wherein the concentration of the peptide is from about 2.5% (w/w) to about 20% (w/w) and wherein the one or more solvents in step a) essentially consists of (and preferably consists of) acetonitrile and water, preferably wherein acetonitrile is present at about 5% (w/w) to about 30% (w/w), or about 5% (w/w) to about 25% (w/w), more preferably about 5% (w/w) to about 20% (w/w), particularly about 15% (w/w) to about 20% (w/w) and most preferably about 19% (w/w); wherein the solution has a pH of: about 5 to about 10, about 6 to about 9, about 7 to about 8.5; and b) lyophilizing the solution.

Preferably in any aspect or embodiment of the disclosure, the solvent in the process consists essentially of, or more particularly or consists of, water and acetonitrile.

In yet another aspect the present disclosure provides peptide powder, preferably dual GLP-1/GIP peptide powder, preferably Tirzepatide or a salt thereof; and most preferably Tirzepatide; produced by any one of the processes described herein

The present disclosure also provides the use of peptide powder produced by the processes described herein in the preparation of pharmaceutical compositions and/or formulations.

The present disclosure also provides peptide powder produced by the processes described herein for use in the preparation of pharmaceutical compositions and/or formulations.

The present disclosure further includes processes for the preparation of the above mentioned pharmaceutical composition comprising a peptide, preferably a dual GLP-1/GIP peptide, preferably Tirzepatide or a salt thereof, and most preferably Tirzepatide, wherein the process comprises combining the peptide powder obtained by any one of the processes described herein with at least one pharmaceutically acceptable excipient.

The peptide powder produced according to the processes described herein and the pharmaceutical compositions or formulations may be used as medicaments, such as for the treatment of type 2 diabetes, overweight or obesity and preferably type 2 diabetes, or for the treatment of: Renal Insufficiency, End Stage Renal Disease, Hepatic Insufficiency, Wolfram Syndrome, Type II Diabetes, Atherosclerosis, Knee Osteoarthritis, Heart Failure With Preserved Ejection Fraction, Obesity and Chronic Kidney Disease, Obstructive Sleep Apnea, Non-Alcoholic Fatty Liver Disease, or Non-alcoholic Steatohepatitis with Type 2 Diabetes.

The peptide powder produced according to the processes described herein and the pharmaceutical compositions or formulations may be used as an appetite suppressant, or for cosmetic weight loss in non-overweight non-obese subjects.

The present disclosure also provides methods for the treatment of type 2 diabetes, overweight or obesity, and preferably type 2 diabetes, or for the treatment of. Renal Insufficiency, End Stage Renal Disease, Hepatic Insufficiency, Wolfram Syndrome, Type II Diabetes, Atherosclerosis, Knee Osteoarthritis, Heart Failure With Preserved Ejection Fraction, Obesity and Chronic Kidney Disease, Obstructive Sleep Apnea, Non-Alcoholic Fatty Liver Disease, or Non-alcoholic Steatohepatitis with Type 2 Diabetes, by administering a therapeutically effective amount of peptide powder produced by any of the processes described herein, or at least one of the above pharmaceutical compositions, to a subject suffering from type 2 diabetes, overweight or obesity and preferably type 2 diabetes.

The present disclosure further provides methods for cosmetic weight loss in non-overweight non-obese subjects by administrating an effective amount of peptide powder produced according to the processes described herein, or pharmaceutical compositions containing the peptide particles produced according to the processes described herein.

The present disclosure also provides uses of peptide powder obtained by the methods of the present disclosure or at least one of the above pharmaceutical compositions, for the manufacture of medicaments for treating type 2 diabetes, overweight or obesity and preferably type 2 diabetes, or for treating: Renal Insufficiency, End Stage Renal Disease, Hepatic Insufficiency, Wolfram Syndrome, Type II Diabetes, Atherosclerosis, Knee Osteoarthritis, Heart Failure With Preserved Ejection Fraction, Obesity and Chronic Kidney Disease, Obstructive Sleep Apnea, Non-Alcoholic Fatty Liver Disease, or Non-alcoholic Steatohepatitis with Type 2 Diabetes.

The present disclosure also provides uses of the peptide powder obtained by the methods of the present disclosure or at least one of the above pharmaceutical compositions, for the manufacturing of medicaments for cosmetic weight loss in non-overweight non-obese subjects.

The present disclosure further relates to processes for preparation of peptide particles, preferably dual GLP-1/GIP peptide particles, preferably Tirzepatide or a salt thereof; and most preferably Tirzepatide; wherein the process comprises: a) providing a solution comprising one or more solvents and a peptide, preferably a dual GLP-1/GIP peptide, preferably Tirzepatide or a salt thereof; and most preferably Tirzepatide; wherein the concentration (w/w) of the peptide is not less than about 2.5%, about 2.5% (w/w) to about 40% w/w), about 2.5% (w/w) to about 35% (w/w), about 2.5% to about 30% (w/w), about 2.5% (w/w) to about 25% (w/w), preferably wherein the concentration of the peptide is from about 2.5% (w/w) to about 20% (w/w), or more particularly about 2.5% (w/w) to about 20% (w/w), or wherein the concentration of the peptide in the solution of step a) is about 3.0 about 20% (w/w), or about 3.5% to about 15% (w/w), or about 3.5% to about 15% (w/w), or about 3.5% to about 15% (w/w), or about 4.0% to about 12% (w/w), or about 4.5% to about 11% (w/w); wherein the solution has a pH of: about 5 to about 10, to about 6 to about 9, about 7 to about 8.5; b) lyophilizing the solution; and c) reducing the particle size of the product of step b), preferably wherein the resulting particles have a PSD characterized by a D(90) value of not more than 120 μm±20%, not more than 100 μm±20%, not more than 80 μm±20%, not more than 60 μm±20%, or a PSD characterized by a D(90) value of: not more than 40 μm 20%, preferably not more than 30 μm±20%, or a PSD characterized by a D(90) value of D(90) of: about 8 μm to about 80 μm, about 10 μm to about 70 μm, about 15 μm to about 65 μm, about 20 μm to about 60 μm, or about 20 to about 40 am.

In any aspect or embodiment the solvents in step a) may be any solvent or a combination of solvents having a freezing point above −70° C. and is suitable for Lyophilization, i.e. sublimes below the triple point in a given minimal pressure, for example 10 pbar. In embodiments the solvents in step a) may be one or more selected from the list consisting of acetic acid, acetone, dimethyl sulfoxide (DMSO), ethanol, water, isopropanol (IPA), methanol, menthol, TFA, and acetonitrile. Preferably, the solvent in step a) may be one or more selected from the group consisting of acetonitrile, IPA, ethanol and water, more preferably the solvent in step a) comprises water. In particular embodiments, the solvent in step a) may comprise water and up to about 60% (w/w), preferably up to about 40% (w/w) of any one or a mixture of acetonitrile, IPA and ethanol. In a specific embodiment the solvent comprises water and about 5% (w/w) to about 30% (w/w) of acetonitrile, more preferably the solvent in step a) comprises water and about 20% (w/w) of acetonitrile. In particularly preferred embodiments, the one or more solvents in step a) essentially consists of (and preferably consists of) acetonitrile and water, preferably wherein acetonitrile is present at about 0.50% (w/w) to about 50% (w/w), about 1.50% (w/w) to about 45% (w/w), about 2.0% (w/w) to about 40% (w/w), about 2.5% (w/w) to about 30% (w/w); or the acetonitrile is present at: about 5% (w/w) to about 30% (w/w), more preferably about 5% (w/w) to about 25% (w/w), more preferably about 5% (w/w) to about 20% (w/w), particularly about 15% (w/w) to about 20% (w/w), and most preferably about 19% (w/w) or at about 0.5% (w/w) to about 50% (w/w), about 1.5% (w/w) to about 45% (w/w), about 2.0% (w/w) to about 40% (w/w), about 2.5% (w/w) to about 30% (w/w); or about 1.0% (w/w) to about 20% (w/w), about 1.5% (w/w) to about 20% (w/w), about 2.0% (w/w) to about 20% (w/w), about 2.5% (w/w) to about 20% (w/w).

In any aspect or embodiment in step a) the pH of the solution may be: about 6 to about 9, more preferably about 7 to about 8.5. If necessary, the pH may be adjusted to within the specified ranges by any suitable basic agent such as but not limited to sodium bicarbonate, ammonium acetate, phosphate buffer, ammonium hydroxide or NaOH. In embodiments the pH is adjusted by ammonium hydroxide, preferably 25% (w/w) solution of ammonium hydroxide or NaOH, to provide the solution in step (a).

In any aspect or embodiment the process of step (a) may be performed at any suitable temperature, typically at a temperature of about 15° C. to about 40° C. and preferably at room temperature.

In any aspect or embodiment the particle size reduction in step c) may be performed for example by any milling technology known in the art or by vibration sieve technology.

In embodiments a jet mill micronizer may be used for milling. In any aspect or embodiment the compressed grinding air (or gas) pressure may be in the range of about 0.1 bar to about 6.0 bar, preferably about 0.1 bar to about 3.0 bar, more preferably about 0.1 bar to about 1.5 bar, most preferably about 0.2 bar to about 1.0 bar. In any aspect or embodiment the compressed feed air (or gas) pressure may be in the about 0.1 bar to about 6.0 bar, preferably about 0.1 bar to about 5.0 bar, more preferably about 1.0 bar to about 4.0 bar, even more preferably about 2.0 bar to about 4.0 bar, and particularly about 3.0 bar. In any aspect or embodiment the feeding rate of the starting particles may range from about 0.5 g/min to about 50 g/min, preferably from about 0.5 g/min to about 10 g/min, more preferably from about 0.5 g/min to about 1.5 g/min and most preferably from about 0.5 g/min to about 1.0 g/min.

Alternatively, a mesh sieve with balls aid may be used for particle size reduction. Vacuum may be applied to improve particle flow through the mesh. In any aspect or embodiment, the balls may be for example ceramic balls, silicate balls or stainless steel balls, preferably ceramic. Preferably the balls are made of zirconium-based ceramics, preferably zirconia. In any aspect or embodiment the size of the balls may vary and may have a diameter of about 0.1 mm to about 10 mm, about 1 to 5 mm, preferably the ball has a diameter of 2 mm. The mesh size may vary from about 10 μm to about 500 μm depending on the desired PSD. Preferably the mesh size is from about 10 to about 300 μm, from about 10 to about 200 μm, from about 10 to about 100 μm, from about 10 to about 80 μm, from about 20 to about 60 μm, more preferably from about 25 μm to about 50 μm, most preferably from about 30 μm to about 50 μm, particularly about 40 μm.

In embodiments the disclosure relates to processes for preparation of peptide particles, preferably dual GLP-1/GIP peptide particles preferably Tirzepatide or a salt thereof; and most preferably Tirzepatide; wherein the process comprises: a) providing a solution comprising one or more solvents and a peptide, preferably a dual GLP-1/GIP peptide, preferably Tirzepatide or a salt thereof, and most preferably Tirzepatide; wherein the concentration (w/w) of the peptide is not less than about 2.5%, about 2.5% (w/w) to about 40% w/w), about 2.5% (w/w) to about 35% (w/w), about 2.5% to about 30% (w/w), about 2.5% (w/w) to about 25% (w/w), preferably wherein the concentration of the peptide is from about 2.5% (w/w) to about 20% (w/w); or more particularly about 2.5% (w/w) to about 20% (w/w), or wherein the concentration of the peptide in the solution of step a) is about 3.0 about 20% (w/w), or about 3.5% to about 15% (w/w), or about 3.5% to about 15% (w/w), or about 3.5% to about 15% (w/w), or about 4.0% to about 12% (w/w), or about 4.5% to about 11% and wherein the solution has a pH of: about 5 to about 10, from about 6 to about 9, from about 7 to about 8.5; b) lyophilizing the solution; and c) reducing the PSD of the particles obtained in step b) by milling or by sieving using a mesh sieve with ball aids, preferably wherein the resulting particles have a PSD characterized by a D(90) value of not more than 120 μm±20%, not more than 100 μm±20%, not more than 80 μm±20%, not more than 60 μm±20%, or a PSD characterized by a D(90) value of: not more than 40 μm±20%, preferably not more than 30 μm±20%; or a PSD characterized by a D(90) value of D(90) of: about 8 μm to about 80 μm, about 10 μm to about 70 μm, about 15 μm to about 65 μm, about 20 μm to about 60 μm, or about 20 to about 40 μm.

In embodiments the disclosure relates to processes for preparation of peptide particles, preferably dual GLP-1/GIP peptide particles, preferably Tirzepatide or a salt thereof; and most preferably Tirzepatide; and more preferably Tirzepatide particles wherein the process comprises a) providing a solution comprising one or more solvents and a peptide, preferably a dual GLP-1/GIP peptide, preferably Tirzepatide or a salt thereof; and most preferably Tirzepatide; wherein the concentration of the peptide is from about 2.5% (w/w) to about 20% (w/w) and wherein the solvents are selected from the list consisting of acetic acid, acetone, dimethyl sulfoxide (DMSO), ethanol, water, isopropanol (IPA), methanol, menthol, TFA, and acetonitrile, preferably, from the group consisting of acetonitrile, IPA, ethanol and water; wherein the solution has a pH of: about 6 to about 9 preferably to about 7 to about 8.5; b) lyophilizing the solution and c) reducing the PSD of the particles obtained in step b) by milling or by sieving using a mesh sieve with ball aids, preferably wherein the resulting particles have a PSD characterized by a D(90) value of not more than 120 μm±20%, not more than 100 μm±20%, not more than 80 μm±20%, not more than 60 μm±20%, or a PSD characterized by a D(90) value of: not more than 40 μm±20%, preferably not more than 30 μm±20%; or a PSD characterized by a D(90) value of D(90) of: about 8 μm to about 80 μm, about 10 μm to about 70 μm, about 15 μm to about 65 μm, about 20 μm to about 60 μm, or about 20 to about 40 μm.

In embodiments the disclosure relates to processes for preparation of peptide particles, preferably dual GLP-1/GIP peptide particles, preferably Tirzepatide or a salt thereof; and most preferably Tirzepatide particles; wherein the process comprises a) providing a solution comprising one or more solvents and a peptide, preferably a dual GLP-1/GIP peptide, preferably Tirzepatide or a salt thereof; and most preferably Tirzepatide; wherein the concentration of the peptide is from about 2.5% (w/w) to about 20% (w/w), and wherein the solvents are selected from the list consisting of acetic acid, acetone, dimethyl sulfoxide (DMSO), ethanol, water, isopropanol (IPA), methanol, menthol, TFA, and acetonitrile, preferably, from the group consisting of acetonitrile, IPA, ethanol and water; wherein the solution has a pH of: about 5 to about 10, about 6 to about 9, about 7 to about 8.5; b) lyophilizing the solution; and c) reducing the PSD of the particles obtained in step b) by milling or by sieving using a mesh sieve with ball aids, preferably wherein the resulting particles have a PSD characterized by a D(90) value of not more than 120 μm±20%, not more than 100 μm±20%, not more than 80 μm±20%, not more than 60 μm±20%, or a PSD characterized by a D(90) value of: not more than 40 μm±20%, preferably not more than 30 μm±20%, or a PSD characterized by a D(90) value of: about 8 μm to about 80 μm, about 10 μm to about 70 μm, about 15 μm to about 65 μm, about 20 μm to about 60 μm, or about 20 to about 40 μm.

In embodiments the disclosure relates to processes for preparation of peptide particles, preferably dual GLP-1/GIP peptide particles, preferably Tirzepatide or a salt thereof; and most preferably Tirzepatide; and more preferably Tirzepatide particles, wherein the process comprises a) providing a solution comprising one or more solvents and a peptide, preferably a dual GLP-1/GIP peptide, preferably Tirzepatide or a salt thereof; and most preferably Tirzepatide; wherein the concentration of the peptide is from about 2.5% (w/w) to about 20% (w/w), and wherein the solvent in step a) comprises water and up to about 60% (w/w), preferably up to about 40% (w/w) of any one or a mixture of acetonitrile, IPA and ethanol; and wherein the solution has a pH of: about 5 to about 10, about 6 to about 9, about 7 to about 8.5; b) lyophilizing the solution and c) reducing the PSD of the particles obtained in step b) by milling or by sieving using a mesh sieve with ball aids, preferably wherein the resulting particles have a PSD characterized by a D(90) value of not more than 120 μm±20%, not more than 100 μm±20%, not more than 80 μm±20%, not more than 60 μm±20%, or wherein the resulting particles have a PSD characterized by a D(90) value of: not more than 40 μm±20%, preferably not more than 30 μm±20%, or a PSD characterized by a D(90) value of: about 8 μm to about 80 μm, about 10 μm to about 70 μm, about 15 μm to about 65 μm, about 20 μm to about 60 μm, or about 20 to about 40 μm.

In embodiments the disclosure relates to processes for preparation of peptide particles, preferably dual GLP-1/GIP peptide particles, preferably Tirzepatide or a salt thereof; and most preferably Tirzepatide particles; wherein the process comprises a) providing a solution comprising one or more solvents and a peptide, preferably a dual GLP-1/GIP peptide, preferably Tirzepatide or a salt thereof; and most preferably Tirzepatide; wherein the concentration of the peptide is from about 2.5% (w/w) to about 20% (w/w) and wherein the solvent in step a) comprises water and up to about 60% (w/w), preferably up to about 40% (w/w) of any one or a mixture of acetonitrile, IPA and ethanol; and wherein the solution has a pH of: about 5 to about 10, about 6 to about 9, about 7 to about 8.5; b) lyophilizing the solution; and c) reducing the PSD of the particles obtained in step b) by milling or by sieving using a mesh sieve with ball aids, preferably wherein the resulting particles have a PSD characterized by a D(90) value of not more than 120 μm±20%, not more than 100 μm 20%, not more than 80 μm±20%, not more than 60 μm±20%, or a PSD characterized by a D(90) value of: not more than 40 μm±20%, preferably not more than 30 μm±20%, or a PSD characterized a D(90) value of: about 8 μm to about 80 μm, about 10 μm to about 70 μm, about 15 μm to about 65 μm, about 20 μm to about 60 μm, or about 20 to about 40 μm.

In embodiments the disclosure relates to processes for preparation of peptide particles, preferably dual GLP-1/GIP peptide particles, preferably Tirzepatide or a salt thereof; and most preferably Tirzepatide; wherein the process comprises a) providing a solution comprising one or more solvents and a peptide, preferably a dual GLP-1/GIP peptide, preferably Tirzepatide or a salt thereof; and most preferably Tirzepatide; the concentration of the peptide is from about 2.5% (w/w) to about 20% (w/w) and wherein the solvent in step a) comprises water and about 5% (w/w) to about 30% (w/w) of acetonitrile, more preferably water and about 20% (w/w) of acetonitrile; wherein the solution has a pH of: about 5 to about 10, about 6 to about 9, about 7 to about 8.5; b) lyophilizing the solution; and c) reducing the PSD of the particles obtained in step b) by milling or by sieving using a mesh sieve with ball aids, preferably wherein the resulting particles have a PSD characterized by a D(90) value of not more than 120 μm±20%, not more than 100 μm±20%, not more than 80 μm±20%, not more than 60 μm±20%, or a PSD characterized by a D(90) value of: not more than 40 μm±20%, preferably not more than 30 μm±20%, preferably wherein the resulting particles have a PSD characterized by a D(90) value of not more than 40 μm±20%, preferably not more than 30 μm±20%, or a PSD characterized by a D(90) value of: about 8 μm to about 80 μm, about 10 μm to about 70 μm, about 15 μm to about 65 μm, about 20 μm to about 60 μm, or about 20 to about 40 μm.

In embodiments the disclosure relates to processes for preparation of peptide particles, preferably dual GLP-1/GIP peptide particles, preferably Tirzepatide or a salt thereof; and most preferably Tirzepatide; wherein the process comprises a) providing a solution comprising one or more solvents and a peptide, preferably a dual GLP-1/GIP peptide, preferably Tirzepatide or a salt thereof; and most preferably Tirzepatide; wherein the concentration of the peptide is from about 2.5% (w/w) to about 13.5% (w/w) and wherein the solvent in step a) comprises water and about 15% (w/w) to about 30% (w/w) of acetonitrile, more preferably water and about 20% (w/w) of acetonitrile; and wherein the solution has a pH of: about 5 to about 10, about 6 to about 9, about 7 to about 8.5; b) lyophilizing the solution; and c) milling the particles obtained in step b) by a jet mill wherein the milling process is characterized by the following:
i) The compressed grinding air (or gas, i.e. the compressed gas or air for grinding) pressure is in the range of about 0.1 bar to about 6.0 bar, preferably about 0.1 bar to about 3.0 bar, more preferably about 0.1 bar to about 1.5 bar, and most preferably about 0.2 bar to about 1.0 bar;
ii) The compressed feed air (or gas) is in the range of about 0.1 bar to about 6.0 bar, preferably about 0.1 bar to about 5.0 bar, more preferably about 1.0 bar to about 4.0 bar, even more preferably about 2.0 bar to about 4.0 bar, and particularly about 3.0 bar; and
iii) The feeding rate of the starting powder ranges from about 0.5 g/min to about 50 g/min, preferably from about 0.5 g/min to about 10 g/min, more preferably from about 0.5 g/min to about 1.5 g/min and most preferably from about 0.5 g/min to about 1.0 g/min;
   and optionally wherein the resulting particles have a PSD characterized by a D(90) value of: not more than 120 μm±20%, not more than 100 μm±20%, not more than 80 μm±20%, not more than 60 μm±20%; or preferably wherein the resulting particles have a PSD characterized by a D(90) value of not more than 40 μm±20%, preferably not more than 30 μm±20% or a PSD characterized by a D(90) value of: about 8 μm to about 80 μm, about 10 μm to about 70 μm, about 15 μm to about 65 μm, about 20 μm to about 60 μm, or about 20 to about 40 μm.

In embodiments the disclosure relates to processes for preparation of peptide particles, preferably dual GLP-1/GIP peptide particles, preferably Tirzepatide or a salt thereof; and most preferably Tirzepatide; wherein the process comprises a) providing a solution comprising one or more solvents and a peptide, preferably a dual GLP-1/GIP peptide, preferably Tirzepatide or a salt thereof; and most preferably Tirzepatide; wherein the concentration of the peptide is from about 2.5% (w/w) to about 20% (w/w) and wherein the solvent in step a) comprises water and about 5% (w/w) to about 30% (w/w) of acetonitrile, more preferably water and about 20% (w/w) of acetonitrile; and wherein the solution has a pH of: to about 5 to about 10, about 6 to about 9, about 7 to about 8.5; b) lyophilizing the solution; and c) milling the particles obtained in step b) by a jet mill wherein the milling process is characterized by the following:
i) compressed grinding air (or gas) pressure is in the range of about 0.1 bar to about 1.5 bar;
ii) compressed feed air (or gas) pressure is in the range of about 0.1 bar to about 6.0 bar, preferably about 0.1 bar to about 5.0 bar, more preferably about 1.0 bar to about 4.0 bar, even more preferably about 2.0 bar to about 4.0 bar, and particularly about 3.0 bar; and
iii) feeding rate of the starting particles ranges from about 0.5 g/min to about 1.5 g/min;
   and optionally wherein the resulting particles have a PSD characterized by a D(90) value of: not more than 120 μm±20%, not more than 100 μm±20%, not more than 80 μm±20%, not more than 60 μm±20%; or preferably wherein the resulting particles have a PSD characterized by a D(90) value of not more than 40 μm±20%, preferably not more than 30 μm±20%, or a PSD characterized by a D(90) value of: about 8 μm to about 80 μm, about 10 μm to about 70 μm, about 15 μm to about 65 μm, about 20 μm to about 60 μm, or about 20 to about 40 μm.

In embodiments the disclosure relates to processes for preparation of peptide particles, preferably dual GLP-1/GIP peptide particles, preferably Tirzepatide or a salt thereof; and most preferably Tirzepatide; wherein the process comprises a) providing a solution comprising one or more solvents and a peptide, preferably a dual GLP-1/GIP peptide, preferably Tirzepatide or a salt thereof; and most preferably Tirzepatide; wherein the concentration of the peptide is from about 2.5% (w/w) to about 13.5% (w/w) and wherein the solvent in step a) comprises water and about 5% (w/w) to about 30% (w/w) of acetonitrile, water and about 5% (w/w) to about 25%, more preferably about 20% (w/w) of acetonitrile; wherein the solution has a pH of: about 5 to about 10, about 6 to about 9 and particularly about 7 to about 8.5; b) lyophilizing the solution; and c) reducing the PSD of the particles obtained in step b) by sieving using a mesh sieve with balls aid wherein the sieving process is characterized by the following:
i) The balls are ceramic balls, silicate balls or stainless steel balls. Preferably the balls are made of zirconium-based ceramic or zirconia.
ii) The balls have a diameter of about 0.1 mm to about 1 cm, preferably the ball has a diameter of 2 mm
iii) The mesh size is from about 10 μm to about 500 μm, preferably the mesh size is 40 μm;
and optionally wherein the resulting particles have a PSD characterized by a D(90) value of: not more than 120 μm±20%, not more than 100 μm±20%, not more than 80 μm±20%, not more than 60 μm±20%; or preferably wherein the resulting particles have a PSD characterized by a D(90) value of not more than 40 μm±20%, preferably not more than 30 μm±20% or a PSD characterized by a D(90) value of: about 8 μm to about 80 μm, about 10 μm to about 70 μm, about 15 μm to about 65 μm, about 20 μm to about 60 μm, or about 20 to about 40 μm

In embodiments the disclosure relates to processes for preparation of peptide particles, preferably dual GLP-1/GIP peptide particles, preferably Tirzepatide or a salt thereof; and most preferably Tirzepatide; wherein the process comprises a) providing a solution comprising one or more solvents and a peptide, preferably a dual GLP-1/GIP peptide, preferably Tirzepatide or a salt thereof; and most preferably Tirzepatide; the concentration of the peptide is from about 2.5% (w/w) to about 20% (w/w) and wherein the solvent in step a) essentially consists of (and preferably consists of) acetonitrile and water, preferably wherein acetonitrile is present at about 5% (w/w) to about 30% (w/w), more preferably about 5% (w/w) to about 25% (w/w), or about 5% (w/w) to about 20% (w/w), more particularly about 15% (w/w) to about 20% (w/w) and most preferably about 19% (w/w); wherein the solution has a pH of: about 5 to about 10, about 6 to about 9, about 7 to about 8.5; b) lyophilizing the solution; and c) reducing the PSD of the particles obtained in step b) by milling or by sieving using a mesh sieve with ball aids, optionally wherein the resulting particles have a PSD characterized by a D(90) value of: not more than 120 μm±20%, not more than 100 μm 20%, not more than 80 μm±20%, not more than 60 μm±20%; or preferably wherein the resulting particles have a PSD characterized by a D(90) value of not more than 40 μm±20%, preferably not more than 30 μm±20%, preferably wherein the resulting particles have a PSD characterized by a D(90) value of not more than 40 μm±20%, preferably not more than 30 μm±20%, or a PSD characterized by a D(90) value of: about 8 μm to about 80 μm, about 10 μm to about 70 μm, about 15 μm to about 65 μm, about 20 μm to about 60 μm, or about 20 to about 40 μm.

In embodiments the disclosure relates to processes for preparation of peptide particles, preferably dual GLP-1/GIP peptide particles, preferably Tirzepatide or a salt thereof; and most preferably Tirzepatide; wherein the process comprises a) providing a solution comprising one or more solvents and a peptide, preferably a dual GLP-1/GIP peptide, preferably Tirzepatide or a salt thereof; and most preferably Tirzepatide; wherein the concentration of the peptide is from about 2.5% (w/w) to about 13.5% (w/w) and wherein the solvent in step a) essentially consists of (and preferably consists of) acetonitrile and water, preferably wherein acetonitrile is present at about 5% (w/w) to about 30% (w/w), or about 5% (w/w) to about 25% (w/w), more preferably about 5% (w/w) to about 20% (w/w), or about 15% (w/w) to about 20% (w/w), and most preferably about 19% (w/w); wherein the solution has a pH of: about 5 to about 10, about 6 to about 9, about 7 to about 8.5; b) lyophilizing the solution; and c) milling the particles obtained in step b) by a jet mill wherein the milling process is characterized by the following:
i) The compressed grinding air (or gas, i.e. the compressed gas or air for grinding) pressure is in the range of about 0.1 bar to about 6.0 bar, preferably about 0.1 bar to about 3.0 bar, more preferably about 0.1 bar to about 1.5 bar, and most preferably about 0.2 bar to about 1.0 bar;
ii) The compressed feed air (or gas) is in the range of about 0.1 bar to about 6.0 bar, preferably about 0.1 bar to about 5.0 bar, more preferably about 1.0 bar to about 4.0 bar, even more preferably about 2.0 bar to about 4.0 bar, and particularly about 3.0 bar; and
iii) The feeding rate of the starting particles ranges from about 0.5 g/min to about 50 g/min, preferably from about 0.5 g/min to about 10 g/min, more preferably from about 0.5 g/min to about 1.5 g/min and most preferably from about 0.5 g/min to about 1.0 g/min;
and optionally wherein the resulting particles have a PSD characterized by a D(90) value of: not more than 120 μm±20%, not more than 100 μm±20%, not more than 80 μm±20%, not more than 60 μm±20%; or preferably wherein the resulting particles have a PSD characterized by a D(90) value of not more than 40 μm±20%, preferably not more than 30 μm±20% or a PSD characterized by a D(90) value of: about 8 μm to about 80 μm, about 10 μm to about 70 μm, about 15 μm to about 65 μm, about 20 μm to about 60 μm, or about 20 to about 40 μm.

In embodiments the disclosure relates to processes for preparation of peptide particles, preferably dual GLP-1/GIP peptide particles, preferably Tirzepatide or a salt thereof; and most preferably Tirzepatide; wherein the process comprises a) providing a solution comprising one or more solvents and a peptide, preferably a dual GLP-1/GIP peptide, preferably Tirzepatide or a salt thereof; and most preferably Tirzepatide; wherein the concentration of the peptide is from about 2.5% (w/w) to about 20% (w/w) and wherein the solvent in step a) essentially consists of (and preferably consists of) acetonitrile and water, preferably wherein acetonitrile is present at about 5% (w/w) to about 30% (w/w), more preferably about 5% (w/w) to about 25% (w/w), and most preferably about 20% (w/w); wherein the solution has a pH of: about 5 to about 10, about 6 to about 9, about 7 to about 8.5; b) lyophilizing the solution; and c) milling the particles obtained in step b) by a jet mill wherein the milling process is characterized by the following:
i) compressed grinding air (or gas) pressure is in the range of about 0.1 bar to about 1.5 bar;
ii) compressed feed air (or gas) pressure is in the range of about 0.1 bar to about 6.0 bar, preferably about 0.1 bar to about 5.0 bar, more preferably about 1.0 bar to about 4.0 bar, even more preferably about 2.0 bar to about 4.0 bar, and particularly about 3.0 bar; and
iii) feeding rate of the starting particles ranges from about 0.5 g/min to about 1.5 g/mins
   and optionally wherein the resulting particles have a PSD characterized by a D(90) value of: not more than 120 μm±20%, not more than 100 μm±20%, not more than 80 μm±20%, not more than 60 μm±20%; or preferably wherein the resulting particles have a PSD characterized by a D(90) value of not more than 40 μm±20%, preferably not more than 30 μm±20%, or a PSD characterized by a D(90) value of: about 8 μm to about 80 μm, about 10 μm to about 70 μm, about 15 μm to about 65 μm, about 20 μm to about 60 μm, or about 20 to about 40 μm.

In embodiments the disclosure relates to processes for preparation of peptide particles, preferably dual GLP-1/GIP peptide particles, preferably Tirzepatide or a salt thereof; and most preferably Tirzepatide; wherein the process comprises a) providing a solution comprising one or more solvents and a peptide, preferably a dual GLP-1/GIP peptide, preferably Tirzepatide or a salt thereof; and most preferably Tirzepatide; wherein the concentration of the peptide is from about 2.5% (w/w) to about 13.5% (w/w) and wherein the solvent in step a) essentially consists of (and preferably consists of) acetonitrile and water, preferably wherein acetonitrile is present at about 5% (w/w) to about 30% (w/w), more preferably about 5% (w/w) to about 25% (w/w), or about 5% (w/w) to about 20% (w/w), particularly about 15% (w/w) to about 20% (w/w), and most preferably about 19% (w/w); wherein the solution has a pH of: about 5 to about 10, about 6 to about 9 and particularly about 7 to about 8.5; b) lyophilizing the solution; and c) reducing the PSD of the particles obtained in step b) by sieving using a mesh sieve with balls aid wherein the sieving process is characterized by the following:
i) The balls are ceramic balls, silicate balls or stainless steel balls. Preferably the balls are made of zirconium-based ceramic or zirconia.
ii) The balls have a diameter of about 0.1 mm to about 1 cm, preferably the ball has a diameter of 2 mm
iii) The mesh size is from about 10 μm to about 500 μm, preferably the mesh size is 40 μm;
   and optionally wherein the resulting particles have a PSD characterized by a D(90) value of: not more than 120 μm±20%, not more than 100 μm±20%, not more than 80 μm±20%, not more than 60 μm±20%; or preferably wherein the resulting particles have a PSD characterized by a D(90) value of not more than 40 μm±20%, preferably not more than 30 μm±20% or a PSD characterized by a D(90) value of: about 8 μm to about 80 μm, about 10 μm to about 70 μm, about 15 μm to about 65 μm, about 20 μm to about 60 μm, or about 20 to about 40 μm

In yet another aspect the disclosure further relates to processes for preparation of peptide particles, preferably dual GLP-1/GIP peptide particles, preferably Tirzepatide or a salt thereof, and most preferably Tirzepatide; wherein the process comprises: providing peptide powder, preferably a dual GLP-1/GIP peptide, preferably Tirzepatide or a salt thereof, and most preferably Tirzepatide; wherein the peptide is obtainable by the process of any aspect or embodiment the present disclosure; and reducing the particle size, optionally wherein the resulting particles have a PSD characterized by a D(90) value of: not more than 120 μm±20%, not more than 100 μm±20%, not more than 80 μm±20%, not more than 60 μm±20%; or preferably wherein the resulting particles have a PSD characterized by a D(90) value of not more than 40 μm±20%, preferably not more than 30 μm±20%, or a PSD characterized by a D(90) value of: about 8 μm to about 80 μm, about 10 μm to about 70 μm, about 15 μm to about 65 μm, about 20 μm to about 60 μm, or about 20 to about 40 μm.

In any aspect or embodiment the particle size reduction in step c) may be performed by any milling technology known in the art. In embodiments a jet mill micronizer may be used. In any aspect or embodiment the pressure grinding air (or gas) may be in the range of about 0.1 bar to about 6.0 bar, preferably about 0.1 bar to about 3.0 bar, more preferably about 0.1 bar to about 1.5 bar, most preferably about 0.2 bar to about 1.0 bar. In any aspect or embodiment the compressed feed air (or gas) pressure may be in the about 0.1 bar to about 6.0 bar, preferably about 0.1 bar to about 5.0 bar, more preferably about 1.0 bar to about 4.0 bar, even more preferably about 2.0 bar to about 4.0 bar, and particularly about 3.0 bar. In any aspect or embodiment the feeding rate of the starting powder may range from about 0.5 g/min to about 50 g/min, preferably from about 0.5 g/min to about 10 g/min, about 0.5 g/min to about 1.5 g/min and most preferably from about 0.5 g/min to about 1.0 g/min.

Alternatively, a mesh sieve with balls aid may be used for particle size reduction. Vacuum may be applied to improve flow of the particles through the mesh. In any aspect or embodiment, the balls may be for example ceramic balls, silicate balls or stainless steel balls, preferably ceramic. Preferably the balls are made of zirconium-based ceramics, preferably zirconia. In any aspect or embodiment the size of the balls may vary and may have a diameter of about 1 mm to about 10 mm, about 1 to about 5 mm, and preferably wherein the ball has a diameter of 2 mm. The mesh size may vary from about 10 μm to about 500 μm depending on the desired PSD. Preferably the mesh size from about 10 to about 500 microns, about 10 to about 300 microns, about 0 to about 200 microns, about 10 to about 100 microns, about 10 to about 80 microns, about 20 to about 60 μm, more preferably from about 25 μm to about 50 μm, most preferably from about 30 μm to about 50 μm, particularly about is 40 μm.

In yet another aspect the present disclosure provides peptide particles, preferably dual GLP-1/GIP peptide particles, preferably Tirzepatide or a salt thereof, and most preferably Tirzepatide; wherein the particles have a PSD characterized by a D(90) value of: not more than 120 μm±20%, not more than 100 μm±20%, not more than 80 μm±20%, not more than 60 μm±20%; or particularly wherein the peptide particles, preferably dual GLP-1/GIP peptide particles, preferably Tirzepatide or a salt thereof; and most preferably Tirzepatide having a PSD characterized by a D(90) value of not more than 40 μm±20%, preferably not more than 30 μm±20%, or a PSD characterized by a D(90) value of: about 8 μm to about 80 μm, about 10 μm to about 70 μm, about 15 μm to about 65 μm, about 20 μm to about 60 μm, or about 20 to about 40 μm, obtainable by any of the processes disclosed herein.

The present disclosure also provides the use of peptide particles, preferably dual GLP-1/GIP peptide particles, preferably Tirzepatide or a salt thereof, and most preferably Tirzepatide; wherein the particles have a PSD characterized by a D(90) value of: not more than 120 μm±20%, not more than 100 μm±20%, not more than 80 μm±20%, not more than 60 μm±20%; or particularly wherein the peptide particles, preferably dual GLP-1/GIP peptide particles, preferably Tirzepatide or a salt thereof; and preferably Tirzepatide having a PSD characterized by a D(90) value of not more than 40 μm±20%, preferably not more than 30 μm±20%, or a PSD characterized by a D(90) value of: about 8 μm to about 80 μm, about 10 μm to about 70 μm, about 15 μm to about 65 μm, about 20 μm to about 60 μm, or about 20 to about 40 μm, obtainable by the processes of the disclosure in the preparation of pharmaceutical compositions and/or formulations.

The present disclosure also provides the peptide particles, preferably dual GLP-1/GIP peptide particles, preferably Tirzepatide or a salt thereof; and most preferably Tirzepatide; wherein the particles have a PSD characterized by a D(90) value of: not more than 120 μm 20%, not more than 100 μm±20%, not more than 80 μm±20%, not more than 60 μm 20%; or particularly wherein the peptide particles, preferably dual GLP-1/GIP peptide particles, preferably Tirzepatide or a salt thereof; and preferably Tirzepatide having a PSD characterized by a D(90) value of not more than 40 μm±20%, preferably not more than 30 μm±20%, or a PSD characterized by a D(90) value of: about 8 μm to about 80 μm, about 10 μm to about 70 μm, about 15 μm to about 65 μm, about 20 μm to about 60 μm, or about 20 to about 40 μm, obtainable by the processes of the disclosure for use in the preparation of pharmaceutical compositions and/or formulations.

The present disclosure further includes processes for the preparation of the above mentioned pharmaceutical composition, or pharmaceutical formulation preferably for oral administration and more preferably wherein the pharmaceutical formulation is a tablet a dual GLP-1/GIP peptide, preferably Tirzepatide or a salt thereof, and most preferably Tirzepatide; wherein the process comprises combining the dual GLP-1/GIP peptide particles, preferably Tirzepatide or a salt thereof, and most preferably Tirzepatide; wherein the particles have a PSD characterized by a D(90) value of: not more than 120 μm±20%, not more than 100 μm±20%, not more than 80 μm±20%, not more than 60 μm±20%; or particularly wherein the peptide particles, preferably dual GLP-1/GIP peptide particles, preferably Tirzepatide or a salt thereof, and preferably Tirzepatide having a PSD characterized by a D(90) value of not more than 40 μm±20%, preferably not more than 30 μm±20% particles, or a PSD characterized by a D(90) value of: about 8 μm to about 80 μm, about 10 μm to about 70 μm, about 15 μm to about 65 μm, about 20 μm to about 60 μm, or about 20 to about 40 μm, obtainable by any of the processes of the present disclosure with at least one pharmaceutically acceptable excipient.

The present disclosure provides dual GLP-1/GIP peptide particles, preferably Tirzepatide or a salt thereof, and most preferably Tirzepatide; wherein the particles have a PSD characterized by a D(90) value of: not more than 120 μm±20%, not more than 100 μm±20%, not more than 80 μm±20%, not more than 60 μm±20%; or particularly wherein the peptide particles, preferably dual GLP-1/GIP peptide particles, preferably Tirzepatide or a salt thereof, and preferably Tirzepatide having a PSD characterized by a D(90) value of not more than 40 μm±20%, preferably not more than 30 μm±20%, or a PSD characterized by a D(90) value of: about 8 μm to about 80 μm, about 10 μm to about 70 μm, about 15 μm to about 65 μm, about 20 μm to about 60 μm, or about 20 to about 40 μm, obtainable by any of the processes described herein and the pharmaceutical compositions or formulations may be used as medicaments, such as for the treatment of patients suffering from type 2 diabetes, overweight or obesity, preferably type 2 diabetes, or for the treatment of patients suffering from: Renal Insufficiency, End Stage Renal Disease, Hepatic Insufficiency, Wolfram Syndrome, Type II Diabetes, Atherosclerosis, Knee Osteoarthritis, Heart Failure With Preserved Ejection Fraction, Obesity and Chronic Kidney Disease, Obstructive Sleep Apnea, Non-Alcoholic Fatty Liver Disease, or Non-alcoholic Steatohepatitis with Type 2 Diabetes.

According to any aspect or embodiment of the process, there is provided a method for producing dual GLP-1/GIP peptide particles, particularly Tirzepatide particles, of any desired particle size distribution. Particularly, the lyophilization process of the present disclosure enables the production the peptide powder which is flowable and hence can be readily subjected to particle size reduction processes as described herein if smaller particles are desired. In particular, the process of the invention can provide lyophilized, peptide powder [i.e. the product obtained after the lyophilization step (b), and prior to any optional particle size reduction step (c)], wherein the flowability is: 0.5 g/sec, at least about 1.0 g/sec, at least about 2.0 g/sec, at least about 3.0 g/sec; or about, at least about 4.0 g/sec, or at least about 5.0 g/sec; or wherein the flowability is: about 0.5 g/sec to about 80 g/sec, about 0.2 g/sec to about 60 g/sec, about 1.0 g/sec to about 70 g/sec, about 2.0 g/sec to about 60 g/sec, about 3.0 g/sec to about 50 g/sec, about 4.0 g/sec to about 30 g/sec, about 4.0 g/sec to about 20 g/sec, about 4.0 g/sec to about 15 g/sec, about 4.0 g/sec to about 12 g/sec, or about 4.5 g/sec to about 12 g/sec, or about 5.0 g/sec to about 12 g/sec, about 5.5 g/sec to about 11 g/sec. This product may be obtainable by any of the lyophilization processes disclosed herein. Advantageously, the surprisingly advantageous flowability of lyophilized product (peptide powder), enables a greater ease of further processing of the peptide powder, for example, in subsequent particle size reduction.

Reduction of particle size by agitative sieving is particularly advantageous for large scale processes. The process of Particle size reduction by Sieving has a very short cycle time in production, it does not involve additional solvents, has low energy consumption and high yield. Therefore, the processes of the present disclosure allow preparation of material having any desired particle size distribution by preparation of the flowable powder and further processing the powder to the required PSD. The present disclosure provides dual GLP-1/GIP peptide particles, preferably Tirzepatide particles having a PSD characterized by a D(90) value of not more than 120 μm±20%, not more than 100 μm±20%, not more than 80 μm 20%, not more than 60 μm±20%, or a PSD characterized by a D(90) value of: not more than 40 μm±20%, preferably not more than 30 μm±20%, a PSD characterized by a D(90) value of: about 8 μm to about 80 μm, about 10 μm to about 70 μm, about 15 μm to about 65 μm, about 20 μm to about 60 μm, or about 20 to about 40 μm, obtainable by any of the processes described herein, and dietary compositions thereof for cosmetic weight loss in non-overweight non-obese subjects.

The present disclosure also provides methods for the treatment of patients with type 2 diabetes, overweight or obesity, preferably type 2 diabetes, or for the treatment of patients with: Renal Insufficiency, End Stage Renal Disease, Hepatic Insufficiency, Wolfram Syndrome, Type II Diabetes, Atherosclerosis, Knee Osteoarthritis, Heart Failure With Preserved Ejection Fraction, Obesity and Chronic Kidney Disease, Obstructive Sleep Apnea, Non-Alcoholic Fatty Liver Disease, or Non-alcoholic Steatohepatitis with Type 2 Diabetes, by administering a therapeutically effective amount of dual GLP-1/GIP peptide particles, preferably Tirzepatide or a salt thereof; and most preferably Tirzepatide; having a PSD characterized by a D(90) value of not more than 120 μm±20%, not more than 100 μm 20%, not more than 80 μm±20%, not more than 60 μm±20%, or a PSD characterized by a D(90) value of: not more than 40 μm±20%, preferably not more than 30 μm±20%, or a PSD characterized by a D(90) value of: about 8 μm to about 80 μm, about 10 μm to about 70 μm, about 15 μm to about 65 μm, about 20 μm to about 60 μm, or about 20 to about 40 μm, obtainable by the processes described herein, or at least one of the above pharmaceutical compositions, to a subject suffering from type 2 diabetes, overweight or obesity, preferably type 2 diabetes, or to a subject suffering from: Renal Insufficiency, End Stage Renal Disease, Hepatic Insufficiency, Wolfram Syndrome, Type II Diabetes, Atherosclerosis, Knee Osteoarthritis, Heart Failure With Preserved Ejection Fraction, Obesity and Chronic Kidney Disease, Obstructive Sleep Apnea, Non-Alcoholic Fatty Liver Disease, or Non-alcoholic Steatohepatitis with Type 2 Diabetes, or otherwise in need of the treatment.

The present disclosure further provides methods for cosmetic weight loss in non-overweight non-obese subjects by administering an effective amount of peptide particles produced according to the processes described herein, or a dietary composition containing the peptide particles produced according to the processes described herein.

The present disclosure also provides uses of dual GLP-1/GIP peptide particles, preferably Tirzepatide or a salt thereof; and most preferably Tirzepatide; having a PSD characterized by a D(90) value of not more than 120 μm±20%, not more than 100 μm±20%, not more than 80 μm±20%, not more than 60 μm±20%, or a PSD characterized by a D(90) value of: not more than 40 μm±20%, preferably not more than 30 μm±20%, or a PSD characterized by a D(90) value of D(90) of: about 8 μm to about 80 μm, about 10 μm to about 70 μm, about 15 μm to about 65 μm, about 20 μm to about 60 μm, or about 20 to about 40 μm, obtained by, or obtainable by the methods of the present disclosure or at least one of the above pharmaceutical compositions, for the manufacture of medicaments for treating e.g., patients with type 2 diabetes, overweight or obesity, and preferably type 2 diabetes, or for the manufacture of a medicament for treating Renal Insufficiency, End Stage Renal Disease, Hepatic Insufficiency, Wolfram Syndrome, Type II Diabetes, Atherosclerosis, Knee Osteoarthritis, Heart Failure With Preserved Ejection Fraction, Obesity and Chronic Kidney Disease, Obstructive Sleep Apnea, Non-Alcoholic Fatty Liver Disease, or Non-alcoholic Steatohepatitis with Type 2 Diabetes.

The present disclosure also provides uses of the peptide particles obtained by the methods of the present disclosure or at least one of the above described dietary compositions for cosmetic weight loss in non-overweight non-obese subjects.

### Methods

### PSD Method:

Instrumentation: Malvern Laser Diffraction Mastersizer 3000, Hydro My cell
Measuring range: 0.01-3500 mcm
Analysis model: Mie Theory
Powder RI: 1.488, Absorption: 1
Dispersant name: Isopar-G
Dispersant RI: 1.42
Speed rate of the flow cell: 2500
Internal Sonication: 1 minute
Recirculation time: 1 minute

### Bulk Density (BD)

Bulk density (BD) was measured according to USP 2014 Method I (616) Bulk Density and Tapped Density of Powders.

SEM micrographs were taken on Phenom Pro, scanning microscope at 10 kV, low current.

Samples were sputtered with gold by Denton Desk V sputter coater.

### Flowability

Instrument: BEP2 Flowability Tester from Copley Scientific, with Funnel Attachment and Manually Operated Stirrer with a nozzle corresponding to aperture size of 25 mm.

### Methodology: Flow Through an Orifice

The test sample was introduced into the funnel and the time required for the entire sample to flow out of the funnel was measure using a suitable stopwatch.

The flow rate results are expressed in terms of mass vs time, i.e., grams per second.

### EXAMPLES

Tirzepatide may be prepared for example according to any method known in the art.

### Example 1: Preparation of Tirzepatide Powder by Lyophilization

Mixture of 22 grams Tirzepatide with 98 grams acetonitrile (ACN) and 393 grams water (to afford a 4.3% w/w solution of Tirzepatide) was stirred in a magnetically stirred vessel at 20° C. The pH of the solution was adjusted to 8.0. The solution was filtered using a 0.2 micron filter and the sample was lyophilized in a Pilot Edwards Lyophilizer minifast according to the process described below.

The solution was charged to a Minifast 10 freeze dryer and then cooled down from 20° C. to −35° C. The frozen material was warmed slowly to 20° C. with reduce pressure of 600 pbar to obtain a primary drying step. After most of the solvent was sublimated, a secondary drying was carried out at 20° C. under a reduced pressure of 200 pbar until complete dryness. The lyophilized material was discharged at room temperature.

BD=0.1 g/ml, flowability=6.1 g/sec

### Example 1A: Preparation of Tirzepatide Powder by Lyophilization

Mixture of 18.4 grams Tirzepatide with 4 grams acetonitrile (ACN) and 145.6 grams water (to afford a 10.95% w/w solution of Tirzepatide) was stirred in a magnetically stirred vessel at 20° C. The pH of the solution was adjusted to 7.3. The solution was filtered using a 0.2 micron filter and the sample was lyophilized in a Pilot Christ Epsilon 2-10D according to the process described below.

The solution was charged to a Epsilon 2-10D freeze dryer and then cooled down from 20° C. to −35° C. The frozen material was warmed slowly to 20° C. with reduce pressure of 600 pbar to obtain a primary drying step. After most of the solvent was sublimated, a secondary drying was carried out at 20° C. under a reduced pressure of 600 pbar until complete dryness. The lyophilized material was discharged at room temperature.

BD=0.13 g/ml, flowability=9.7 g/sec

### Example 1B: Preparation of Tirzepatide Powder by Lyophilization

Mixture of 2 grams Tirzepatide with 4 grams acetonitrile (ACN) and 394 grams water (to afford a 0.5% w/w solution of Tirzepatide) was stirred in a magnetically stirred vessel at 20° C. The pH of the solution was 7.3. The solution was filtered using a 0.2 micron filter and the sample was lyophilized in a Pilot Christ Epsilon 2-10D according to the process described below.

The solution was charged to a Epsilon 2-10D freeze dryer and then cooled down from 20° C. to −35° C. The frozen material was warmed slowly to 20° C. with reduce pressure of 600 pbar to obtain a primary drying step. After most of the solvent was sublimated, a secondary drying was carried out at 20° C. under a reduced pressure of 600 pbar until complete dryness. The lyophilized material was discharged at room temperature. BD=0.007 g/ml, flowability: non flowable

### Example 1C: Preparation of Tirzepatide Powder by Lyophilization

A solution of Tirzepatide (22 grams), acetonitrile (98 grams) and water (393 grams) was stirred at 20° C. The pH of the solution was adjusted to 8.0 with 0.1M NaOH solution. After filtration using a 0.2 micron filter, the solution was lyophilized in a Pilot Edwards Lyophilizer minifast according to the process described below.

The solution was charged to a Minifast 10 freeze dryer and then cooled down from 20° C. to −35° C. The frozen material was warmed slowly to 20° C. with reduced pressure of 600 pbar to obtain a primary drying step. After most of the solvent was sublimated, a secondary drying was carried out at 20° C. under reduced pressure (200 pbar) until complete dryness. The lyophilized material was discharged at room temperature.

### Example 2: Preparation of Micronized Tirzepatide by Milling Using Jet Mill

1.5 grams of Tirzepatide powder (prepared according to Example 1, BD=0.1 g/ml) was fed to a 50 mm jet mill using vibration feeder with a feeding rate of 1 g/min, compressed grinding air at a pressure of 0.2 bar and compressed feed air pressure of 3.0 bar. The output was 0.5 grams of micronized powder with particle sizes of d(0.9)=21.9 μm. Yield was 33%.

### Example 3: Preparation of Tirzepatide by Sieving d(0.9)=31 μm

1.0 gram of Tirzepatide powder (prepared according to Example 1, BD=0.1 g/ml) was passed through a 20 micron mesh sieve with ceramic ball sieving aid (Zirmil ball, diameter 2 mm), using vibration. The output was 0.96 grams of powder with particles size of d(0.9)=30.9 μm (yield=96%).

### Example 3A: Preparation of Tirzepatide by Sieving d(0.9)=59 μm

1.5 grams of Tirzepatide powder (prepared according to Example 1A, BD=0.13 g/ml) was passed through a 44 micron mesh sieve with ceramic ball sieving aid (Zirmil ball, diameter 2 mm), using vibration. The output was 1.4 grams of powder with particles size of d(0.9)=59.1 μm (yield=93%).

### Example 3B: Preparation of Tirzepatide by sieving d(0.9)=128 μm

0.35 grams of Tirzepatide powder (prepared according to Example 1A, BD=0.13 g/ml) was passed through a 106 micron mesh sieve with ceramic ball sieving aid (Zirmil ball, diameter 2 mm), using vibration. The output was 0.34 grams of powder with particles size of d(0.9)=128.0 μm (yield=97%).

Further aspects and embodiments of the disclosure are described in the following numbered clauses
1. A process for preparation of a peptide powder wherein the process comprises: a) providing a solution comprising one or more solvents and a peptide, wherein the concentration (w/w) of the peptide is not less than about 2.5%, preferably wherein the concentration of the peptide is from about 2.5% (w/w) to about 20% (w/w); wherein the solution has a pH of: about 5 to about 10, about 6 to about 9 or about 7 to about 8.5; and b) lyophilizing the solution.
2. A process according to clause 1, wherein the concentration of the peptide in the solution of step a) is from: about 2.5% (w/w) to about 40% w/w), about 2.5% (w/w) to about 35% (w/w), about 2.5% to about 30% (w/w), about 2.5% (w/w) to about 25% (w/w), or about 2.5% (w/w) to about 20% (w/w), or about 3.0 about 20% (w/w), or about 3.5% to about 15% (w/w), or about 3.5% to about 15% (w/w), or about 3.5% to about 15% (w/w), or about 4.0% to about 12% (w/w), or about 4.5% to about 11% (w/w).
3. A process according to clause 1, wherein the concentration of the peptide in the solution of step a) is from: about 2.5% (w/w) to about 40% w/w), about 2.5% (w/w) to about 350% (w/w), about 2.5% to about 30% (w/w), about 2.5% (w/w) to about 25% (w/w), or about 2.5% (w/w) to about 20% (w/w).
4. A process according to clause 1, 2 or 3, wherein the one or more solvents are selected from the group consisting of acetic acid, acetone, dimethyl sulfoxide (DMSO), ethanol, water, isopropanol (IPA), methanol, menthol, TFA, and acetonitrile, preferably from the group consisting of acetonitrile, IPA, ethanol and water.
5. A process according to clause 1, 2, 3, or 4, wherein the one or more solvents comprises water.
6. A process according to any of clauses 1, 2, 3, 4, or 5, wherein the one or more solvents comprises water and up to about 60% (w/w), preferably up to about 40% (w/w) of any one or a mixture of acetonitrile, IPA and ethanol.
7. A process according to any of clauses 1, 2, 3, 4, 5, or 6, wherein the one or more solvents consists essentially of, and preferably consists of, acetonitrile and water, more preferably wherein acetonitrile is present at a concentration of: about 0.5% (w/w) to about 50% (w/w), about 1.5% (w/w) to about 45% (w/w), about 2.0% (w/w) to about 40% (w/w), about 2.5% (w/w) to about 30% (w/w); or about 5% (w/w) to about 30% (w/w), more preferably about 5% (w/w) to about 25% (w/w), or about 5% (w/w) to about 20% (w/w), particularly about 15 to about 20% (w/w), and especially about 19%; or about 1.0% (w/w) to about 25% (w/w), about 1.5% (w/w) to about 25% (w/w), about 2.0% (w/w) to about 25% (w/w), about 2.5% (w/w) to about 25% (w/w); or about 1.0% (w/w) to about 20% (w/w), about 1.5% (w/w) to about 20% (w/w), about 2.0% (w/w) to about 20% (w/w), about 2.5% (w/w) to about 20% (w/w).
8. A process according to any of clauses 1, 2, 3, 4, 5, 6, or 7, wherein the one or more solvents consists essentially of, and preferably consists of, acetonitrile and water, more preferably wherein acetonitrile is present at a concentration of: about 5% (w/w) to about 30% (w/w), more preferably about 5% (w/w) to about 25% (w/w), or about 5% (w/w) to about 20% (w/w), particularly about 15 to about 20% (w/w), and especially about 19%.
9. A process according to any of clauses 1, 2, 3, 4, 5, 6, 7, or 8, wherein the pH of the solution in step (a) is: about 6 to about 9, or about 6 to about 8.5, preferably to about 7 to about 8, more preferably to about 7.3 or about 8.0.
10. A process according to any of clauses 1, 2, 3, 4, 5, 6, 7, 8, or 9, wherein the pH of the solution in step (a) is: about 6 to about 9, preferably to about 7 to about 8, more preferably about 7.3 or about 8.0.
11. A process according to any of clauses 1, 2, 3, 4, 5, 6, 7, 8, 9 or 10, wherein the pH of the solution is adjusted if necessary using a base, preferably selected from the sodium bicarbonate, ammonium acetate, phosphate buffer, ammonium hydroxide or NaOH, preferably wherein the pH is adjusted by addition of ammonium hydroxide or NaOH, more preferably by addition of a 25% (w/w) solution of ammonium hydroxide or NaOH.
12. A process according to any of clauses 1, 2, 3, 4, 5, 6, 7, 8, 9, 10, or 11, wherein step (a) is performed at a temperature of about 15° C. to about 40° C., or about 15° C. to about 30° C., and preferably at room temperature; or wherein step (a) is performed at a temperature of: about 15° C. to about 35° C., about 18° C. to about 25° C., or about 18° C. to about 22° C., or about 20° C.
13. A process according to any of clauses 1, 2, 3, 4, 5, 6, 7, 8, 9, 10, 11, or 12, wherein step (a) is performed at a temperature of about 15° C. to about 40° C., and preferably at room temperature.
14. A process according to any of clauses 1, 2, 3, 4, 5, 6, 7, 8, 9, 10, 11, 12, or 13, wherein the solution in step (a) is filtered prior to step (b).
15. A process according to any of clauses 1, 2, 3, 4, 5, 6, 7, 8, 9, 10, 11, 12, 13, or 14, wherein step (b) is carried at a temperature of: about −45° C. to about 30° C., about −40° C. to about 25° C., about −40° C. to about 25° C., or about −35° C. to about 20° C.
16. A process according to any of clauses 1, 2, 3, 4, 5, 6, 7, 8, 9, 10, 11, 12, 13, 14, or 15, wherein the freeze dried product is subjected to a further drying step.
17. A process according to clause 16, wherein the further drying step is carried out at a temperature of: about 15° C. to about 35° C., about 18° C. to about 25° C., or about 18° C. to about 22° C., or about 20° C.
18. A process according to clause 16 or clause 17, wherein the further drying step is carried out at reduced pressure, preferably at a pressure of: about 100 pbar to about 800 pbar, about 150 pbar to about 700 pbar, about 180 to about 650 pbar, or about 200 to about 600 pbar.
19. A process according to any of clauses 1, 2, 3, 4, 5, 6, 7, 8, 9, 10, 11, 12, 13, 14, 15, 16, 17, or 18, wherein the process further comprises: c) reducing the particle size of the product of step (b).
20. A process according to clause 19, wherein the product from step c) has a PSD characterized by a D(90) value of not more than 120 μm±20%, not more than 100 μm 20%, not more than 80 μm±20%, not more than 60 μm±20%, or a PSD characterized by a D(90) value of: not more than 40 μm±20%, preferably not more than 30 μm±20%, or a PSD characterized by a D(90) value of D(90) of: about 8 μm to about 80 μm, about 10 μm to about 70 μm, about 15 μm to about 65 μm, about 20 μm to about 60 μm, or about 20 to about 40 μm.
21. A process according to clause 19 or 20, wherein step (c) comprises milling and/or sieving.
22. A process according to any of clauses 19, 20, or 21, wherein the particle size reduction is performed using a jet mill micronizer.
23. A process according to clause 22, wherein the compressed grinding air (or gas) pressure in the jet mill may be in the range of about 0.1 bar to about 6.0 bar, preferably about 0.1 bar to about 3.0 bar, more preferably about 0.1 bar to about 1.5 bar, most preferably about 0.3 bar to about 1.0 bar.
24. A process according to clause 22 or 23, wherein the feeding air (or gas) pressure may be in the about 0.1 bar to about 6.0 bar, preferably about 0.1 bar to about 5.0 bar, more preferably about 1.0 bar to about 4.0 bar, even more preferably about 2.0 bar to about 4.0 bar, and particularly about 3.0 bar.
25. A process according to any of clauses 22, 23, or 24, wherein the powder feeding rate is from about 0.5 g/min to about 50 g/min, preferably from about 0.5 g/min to about 10 g/min, more preferably from about 0.5 g/min to about 1.5 g/min and most preferably from about 0.5 g/min to about 1.0 g/min.
26. A process according to any of clauses 19, 20, or 21, wherein the particle size reduction is performed by sieving using a mesh sieve and balls as a sieving aid.
27. A process according to clause 26, wherein the balls are ceramic balls, silicate balls, or stainless steel balls, preferably ceramic balls.
28. A process according to clause 27, wherein the ceramic balls are zirconium-based ceramic balls, and preferably made of zirconia.
29. A process according to any of clauses 26, 27, or 28, wherein the balls have a diameter of about 1 mm to about 10 mm, about 1 mm to about 5 mm, or about 2 mm.
30. A process according to any of clauses 26, 27, 28, or 29, wherein the mesh size of the mesh sieve is from about 20 to about 60 μm, more preferably from about 25 μm to about 50 μm, most preferably from about 30 μm to about 50 μm, particularly about 40 μm.
31. A process according to any of clauses 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30, wherein the flowability of the lyophilized product obtained in step (b) is: at least about 0.5 g/sec, at least about 1.0 g/sec, at least about 2.0 g/sec, at least about 3.0 g/sec; or about, at least about 4.0 g/sec, or at least about 5.0 g/sec; or wherein the flowability is: about 0.5 g/sec to about 80 g/sec, about 0.2 g/sec to about 60 g/sec, about 1.0 g/sec to about 70 g/sec, about 2.0 g/sec to about 60 g/sec, about 3.0 g/sec to about 50 g/sec, about 4.0 g/sec to about 30 g/sec, about 4.0 g/sec to about 20 g/sec, about 4.0 g/sec to about 15 g/sec, about 4.0 g/sec to about 12 g/sec, or about 4.5 g/sec to about 12 g/sec, or about 5.0 g/sec to about 12 g/sec, about 5.5 g/sec to about 11 g/sec, as measured according to the method described in the application.
32. A process according to any of Claims 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31, wherein the peptide is a dual GLP-1/GIP peptide; preferably Tirzepatide or a salt thereof, and most preferably Tirzepatide.
33. A peptide powder, or peptide particles produced by any one of the processes of the preceding clauses, preferably wherein the peptide is a dual GLP-1/GIP peptide, preferably Tirzepatide or a salt thereof, and most preferably Tirzepatide.
34. A peptide powder, or peptide particles obtainable by any of the processes of clauses 19, 20, 21, 22, 23, 24, 25, 26, 27, 28, 29, 30, 31, 32, or 33, preferably wherein the peptide is a dual GLP-1/GIP peptide, preferably Tirzepatide or a salt thereof, and most preferably Tirzepatide.
35. Lyophilized, peptide powder or produced by any of the processes of clauses 1, 2, 3, 4, 5, 6, 7, 8, 9, 10, 11, 12, 13, 14, 15, 16, 17, or 18, wherein the flowability is: 0.5 g/sec, at least about 1.0 g/sec, at least about 2.0 g/sec, at least about 3.0 g/sec; or about, at least about 4.0 g/sec, or at least about 5.0 g/sec; or wherein the flowability is: about 0.5 g/sec to about 80 g/sec, about 0.2 g/sec to about 60 g/sec, about 1.0 g/sec to about 70 g/sec, about 2.0 g/sec to about 60 g/sec, about 3.0 g/sec to about 50 g/sec, about 4.0 g/sec to about 30 g/sec, about 4.0 g/sec to about 20 g/sec, about 4.0 g/sec to about 15 g/sec, about 4.0 g/sec to about 12 g/sec, or about 4.5 g/sec to about 12 g/sec, or about 5.0 g/sec to about 12 g/sec, about 5.5 g/sec to about 11 g/sec, as measured according to the method described in the application.
36. A pharmaceutical composition, pharmaceutical formulation, or dietary composition, preferably a pharmaceutical composition or pharmaceutical formulation, comprising peptide particles according to any of clauses 33, 34 or 35.
37. A peptide powder, or peptide particles according to any of clauses 33, 34, or 35, for use in the preparation of a pharmaceutical composition, pharmaceutical formulation, or a dietary composition, preferably for use in the preparation of a pharmaceutical compositions or a pharmaceutical formulation.
38. A process for the preparation of a pharmaceutical composition, a pharmaceutical formulation, or a dietary composition as defined in clause 36, comprising combining the peptide particles obtained by any one of the processes of clauses 1-33, 34 or 35, with at least one pharmaceutically acceptable excipient.
39. Dual GLP-1/GIP peptide particles according to clause 33, 34 or 35, a pharmaceutical composition or pharmaceutical formulation thereof according to clause 36, for use in the treatment of type 2 diabetes, overweight or obesity, and preferably type 2 diabetes, or for use in the treatment of Renal Insufficiency, End Stage Renal Disease, Hepatic Insufficiency, Wolfram Syndrome, Type II Diabetes, Atherosclerosis, Knee Osteoarthritis, Heart Failure With Preserved Ejection Fraction, Obesity and Chronic Kidney Disease, Obstructive Sleep Apnea, Non-Alcoholic Fatty Liver Disease, or Non-alcoholic Steatohepatitis with Type 2 Diabetes, particularly wherein the peptide is Tirzepatide or a salt thereof; and most preferably Tirzepatide.
40. Dual GLP-1/GIP peptide particles according to clause 33, 34 or 35, a pharmaceutical composition or pharmaceutical formulation thereof according to clause 36, for use in the treatment of type 2 diabetes, overweight or obesity, and preferably type 2 diabetes, particularly wherein the peptide is Tirzepatide or a salt thereof, and most preferably Tirzepatide.
41. Dual GLP-1/GIP peptide particles according to clause 33, 34 or 35, or dietary composition according to clause 36, for cosmetic weight loss in non-overweight non-obese subjects, particularly wherein the peptide is Tirzepatide or a salt thereof, and most preferably Tirzepatide.
42. A method for cosmetic weight loss in non-overweight non-obese subjects comprising by administering an effective amount of dual GLP-1/GIP peptide particles according to clause 33, 34, or 35 or dietary composition according to clause 36, particularly wherein the peptide is Tirzepatide or a salt thereof, and most preferably Tirzepatide.

1. A process for preparation of a peptide powder wherein the process comprises: a) providing a solution comprising one or more solvents and a peptide, wherein the concentration (w/w) of the peptide is: not less than about 2.5% or from about 2.5% (w/w) to about 20% (w/w); wherein the solution has a pH of: about 5 to about 10, about 6 to about 9 or about 7 to about 8.5; and b) lyophilizing the solution.

2. The process according to claim 1, wherein the concentration of the peptide in the solution of step a) is from: about 2.5% (w/w) to about 40% w/w), about 2.5% (w/w) to about 35% (w/w), about 2.5% to about 30% (w/w), about 2.5% (w/w) to about 25% (w/w), or about 2.5% (w/w) to about 20% (w/w), about 3.0 about 20% (w/w), about 3.5% to about 15% (w/w), about 3.5% to about 15% (w/w), about 3.5% to about 15% (w/w), about 4.0% to about 12% (w/w), or about 4.5% to about 11% (w/w).

3. The process according to claim 1, wherein the concentration of the peptide in the solution of step a) is from: about 2.5% (w/w) to about 40% w/w), about 2.5% (w/w) to about 35% (w/w), about 2.5% to about 30% (w/w), about 2.5% (w/w) to about 25% (w/w), or about 2.5% (w/w) to about 20% (w/w).

4. The process according to claim 1, wherein the one or more solvents are selected from the group consisting of acetic acid, acetone, dimethyl sulfoxide (DMSO), ethanol, water, isopropanol (IPA), methanol, menthol, TFA, and acetonitrile, preferably from the group consisting of acetonitrile, IPA, ethanol and water.

5. The process according to claim 1, wherein the one or more solvents comprises water.

6. The process according to claim 1, wherein the one or more solvents comprises water and up to about 60% (w/w) or about 40% (w/w) of any one or a mixture of acetonitrile, IPA, or ethanol.

7. The process according to claim 1, wherein the one or more solvents consists essentially of acetonitrile and water, and wherein acetonitrile is present at a concentration of: about 0.5% (w/w) to about 50% (w/w), about 1.5% (w/w) to about 45% (w/w), about 2.0% (w/w) to about 40% (w/w), about 2.5% (w/w) to about 30% (w/w); about 5% (w/w) to about 30% (w/w), more preferably about 5% (w/w) to about 25% (w/w), about 5% (w/w) to about 20% (w/w), particularly about 15 to about 20% (w/w), and especially about 19%; about 1.0% (w/w) to about 25% (w/w), about 1.50% (w/w) to about 25% (w/w), about 2.0% (w/w) to about 25% (w/w), about 2.5% (w/w) to about 25% (w/w); about 1.0% (w/w) to about 20% (w/w), about 1.5% (w/w) to about 20% (w/w), about 2.0% (w/w) to about 20% (w/w), or about 2.5% (w/w) to about 20% (w/w).

8. The process according to claim 1, wherein the one or more solvents consists essentially of acetonitrile and water, and wherein acetonitrile is present at a concentration of: about 5% (w/w) to about 30% (w/w), about 5% (w/w) to about 25% (w/w), about 5% (w/w) to about 20% (w/w), about 15 to about 20% (w/w), or about 19%.

9. The process according to claim 1, wherein the pH of the solution in step (a) is: about 6 to about 9, about 6 to about 8.5, to about 7 to about 8, or to about 7.3 or about 8.0.

10. The process according to claim 1, wherein the solution in step (a) is filtered prior to step (b).

11. The process according to claim 1, wherein the lyophilized product of step (b) is subjected to a further drying step.

12. The process according to claim 1, wherein the process further comprises: c) reducing the particle size of the lyophilized product of step (b).

13. The process according to claim 12, wherein the product from step c) has a PSD characterized by a D(90) value of: not more than 120 μm±20%, not more than 100 μm±20%, not more than 80 μm±20%, not more than 60 μm±20%, not more than 40 μm±20%, not more than 30 μm±20%, about 8 μm to about 80 μm, about μm to about 70 μm, about 15 μm to about 65 μm, about 20 μm to about 60 μm, or about 20 to about 40 μm.

14. The process according to claim 12, wherein step (c) comprises milling and/or sieving.

15. The process according to claim 12, wherein the particle size reduction is performed using a jet mill micronizer.

16. The process according to claim 15, wherein the compressed grinding gas pressure in the jet mill micronizer is in the range of about 0.1 bar to about 6.0 bar, about 0.1 bar to about 3.0 bar, about 0.1 bar to about 1.5 bar, or about 0.3 bar to about 1.0 bar.

17. The process according to claim 15, wherein the feeding gas pressure in the jet mill micronizer is from: about 0.1 bar to about 6.0 bar, about 0.1 bar to about 5.0 bar, about 1.0 bar to about 4.0 bar, about 2.0 bar to about 4.0 bar, or about 3.0 bar.

18. The process according to claim 15, wherein the powder feeding rate is from about 0.5 g/min to about 50 g/min, preferably from about 0.5 g/min to about 10 g/min, more preferably from about 0.5 g/min to about 1.5 g/min and most preferably from about 0.5 g/min to about 1.0 g/min.

19. The process according to claim 12, wherein the particle size reduction is performed by sieving using a mesh sieve and balls as a sieving aid.

20. The process according to claim 19, wherein the balls are ceramic balls, silicate balls, or stainless steel balls.

21. The process according to claim 20, wherein the ceramic balls are zirconium-based ceramic balls made of zirconia.

22. The process according to claim 19, wherein the balls have a diameter of about 1 mm to about 10 mm, about 1 mm to about 5 mm, or about 2 mm.

23. The process according to claim 19, wherein the mesh size of the mesh sieve is from about 20 to about 60 μm, from about 25 μm to about 50 μm, from about 30 μm to about 50 μm, or about 40 μm.

24. The process according to claim 1, wherein the flowability of the lyophilized product obtained in step (b) is: at least about 0.5 g/sec, at least about 1.0 g/sec, at least about 2.0 g/sec, at least about 3.0 g/sec; or about, at least about 4.0 g/sec, or at least about 5.0 g/sec; or wherein the flowability is: about 0.5 g/sec to about 80 g/sec, about 0.2 g/sec to about 60 g/sec, about 1.0 g/sec to about 70 g/sec, about 2.0 g/sec to about 60 g/sec, about 3.0 g/sec to about 50 g/sec, about 4.0 g/sec to about 30 g/sec, about 4.0 g/sec to about 20 g/sec, about 4.0 g/sec to about 15 g/sec, about 4.0 g/sec to about 12 g/sec, or about 4.5 g/sec to about 12 g/sec, or about 5.0 g/sec to about 12 g/sec, or about 5.5 g/sec to about 11 g/sec.

25. The process according to claim 1, wherein the peptide is a dual GLP-1/GIP peptide including at least one of Tirzepatide or a salt thereof.

26. A peptide powder, or peptide particles produced by a process of claim 1, wherein the peptide powder or peptide particles is a dual GLP-1/GIP peptide including at least one of Tirzepatide or a salt thereof.

27. A peptide powder, or peptide particles obtainable by of claim 12, wherein the peptide is a dual GLP-1/GIP peptide including at least one of Tirzepatide or a salt thereof.

28. A lyophilized peptide powder produced by a process of claim 1, wherein the flowability is: 0.5 g/sec, at least about 1.0 g/sec, at least about 2.0 g/sec, at least about 3.0 g/sec; or about, at least about 4.0 g/sec, or at least about 5.0 g/sec; or wherein the flowability is: about 0.5 g/sec to about 80 g/sec, about 0.2 g/sec to about 60 g/sec, about 1.0 g/sec to about 70 g/sec, about 2.0 g/sec to about 60 g/sec, about 3.0 g/sec to about 50 g/sec, about 4.0 g/sec to about 30 g/sec, about 4.0 g/sec to about 20 g/sec, about 4.0 g/sec to about 15 g/sec, about 4.0 g/sec to about 12 g/sec, or about 4.5 g/sec to about 12 g/sec, or about 5.0 g/sec to about 12 g/sec, or about 5.5 g/sec to about 11 g/sec.

29. A pharmaceutical composition, pharmaceutical formulation, or dietary composition comprising powder or peptide particles according to claim 26.

30. (canceled)

31. A process for the preparation of a pharmaceutical composition, a pharmaceutical formulation, or a dietary composition, comprising combining the peptide powder or the peptide particles according to claim 26, with at least one pharmaceutically acceptable excipient to form the pharmaceutical composition, the pharmaceutical formulation, or the dietary composition.

32. A method of treatment comprising administering to a subject in need thereof an effective amount of the pharmaceutical composition, the pharmaceutical formulation, or the dietary composition according to claim 29, wherein the treatment is for at least one of type 2 diabetes, overweight or obesity, Renal Insufficiency, End Stage Renal Disease, Hepatic Insufficiency, Wolfram Syndrome, Type II Diabetes, Atherosclerosis, Knee Osteoarthritis, Heart Failure With Preserved Ejection Fraction, Obesity and Chronic Kidney Disease, Obstructive Sleep Apnea, Non-Alcoholic Fatty Liver Disease, or Non-alcoholic Steatohepatitis with Type 2 Diabetes.

33. A method of treatment comprising administering to a subject in need thereof an effective amount of the the pharmaceutical composition, the pharmaceutical formulation, or the dietary composition according to claim 29, wherein the treatment is for at least one of type 2 diabetes or overweight or obesity.

34. (canceled)

35. A method for cosmetic weight loss in non-overweight non-obese subjects comprising by administering an effective amount, the pharmaceutical composition, the pharmaceutical formulation, or dietary composition according to claim 29 to a subject in need thereof.
